(19) United States
(12) Patent Application Publication
(10) Pub. No.: US 2002/0132812 A1
(43) Pub. Date: Sep. 19, 2002

(54) Oral hypoglycaemic agents

(76) Inventors: Henrik Sune Andersen, Lyngby (DK); Palle Jakobsen, Vaerlose (DK); Peter Madsen, Bagsvaerd (DK)

Correspondence Address: Reza Green, Esq., Novo Nordisk of North America, Inc., Suite 6400, 405 Lexington Avenue, New York, NY, 10174-6401, US

(21) Appl. No.: 10/039,723

(22) Filed: Nov. 9, 2001

Related U.S. Application Data
(60) Provisional application No. 60/252,487, filed on Nov. 21, 2000.

(30) Foreign Application Priority Data
Nov. 10, 2000 (DK) PA 2000 01684
Sep. 4, 2001 (DK) PA 2001 01295

(51) Int. Cl. C 07D 4 3/02; A61K 31/506; A61K 31/501; A61K 31/4709; A61K 31/4439

(52) U.S. Cl. 514/252.02; 514/252.05; 514/307; 514/312; 514/341; 514/365; 514/397; 544/238; 544/333; 546/148; 546/176; 546/272.7; 548/312.7

(57) ABSTRACT

Compounds of formula (I) which are optionally substituted 2-(ω,ω-diarylalkyl)-4,5-dihydro-1H-imidazoles and 2-(ω,ω-diarylalkyl)-1,4,5,6-tetrahydropyrimidines and salts thereof with inorganic and organic acids have interesting pharmacological properties. Thus, the compounds are useful in the treatment of type 2 diabetes.

### CROSS-REFERENCE TO RELATED APPLICATIONS

This application claims the benefit under 35 U.S.C. 119 of Danish applications PA 2000 01684 and PA 2001 01295 filed Nov. 10, 2000 and Sep. 4, 2001 respectively, and of U.S. application No. 60/252,487 filed Nov. 21, 2000, the contents of which are hereby fully incorporated by reference.

### FIELD OF THE INVENTION

The present invention relates to novel substituted 1,3-diazaheterocyclic compounds of the general formula (I) described below and salts thereof, to methods for their preparation, to pharmaceutical compositions containing them, to the use of the compounds in the manufacture of a medicament and to their use in the treatment of type 2 diabetes,

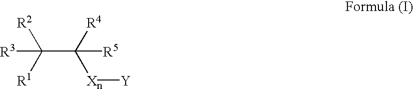

wherein Y is selected from the following ring systems

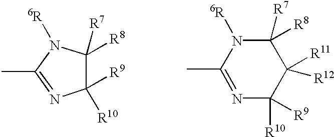

and R¹, R², R³, R⁴ R⁵, R⁶, R⁷, R⁸, R⁹, R¹⁰, R¹¹, R¹², X, and n are defined below.

### PRIOR ART

Certain 1,3-diazaheterocyclic compounds of the general formula (I) are described in literature. Thus, J O Jilek et al. J Chem Soc (1950) 188 describe 2-(2,2-diphenylethyl)-4,5-dihydro-1H-imidazole. Jilek et al. propose that the compound may have antihistaminic action. In U.S. Pat. No. 4,138,491 (Daiichi Seiyaku) it is contemplated that the same compound may be useful in the treatment of diabetes.

N Sperber et al. J Am Chem Soc 75 (1953) 2986-2988 describe 2-[2-phenyl-2-(2-pyridyl)]ethyl-2-imidazoline and 2-[2-(4-chlorophenyl)-2-(2-pyridyl)]ethyl-2-imidazoline. Sperber et al. (loc. cit. and in U.S. Pat. No. 2,604,473 (Schering Corporation)) propose that the compounds may be useful as histamine antagonists. In U.S. Pat. No. 4,138,491 (Daiichi Seiyaku) it is proposed that 2-[2-phenyl-2-(2-pyridyl)]ethyl-2-imidazoline may be useful in the treatment of diabetes.

U.S. Pat. No. 2,752,358 (Farbwerke Hoechst A G) describes 2-[2-(indol-3-yl)-2-phenyl]ethyl-2-imidazoline; 2-[2-(2-methylindol-3-yl)-2-phenyl]ethyl-2-imidazoline; 2-[2-(indol-3-yl)-2-(3-methoyxphenyl)]ethyl-2-imidazoline; 2-[2-(2-methylindol-3-yl)-2-(3-methoxyphenyl)]ethyl-2-imidazoline and 2-[2-(5-chloro-2-methylindol-3-yl)-2-(3-methoxyphenyl)]ethyl-2-imidazoline. It is proposed that the compounds have diuretic action, sympaticolytic action and may be useful in the treatment of headache, in particular migraine.

GB 1,008,649 (Chinoin Gyogyszer) describes 2-[2-(4-chlorophenyl)-2-phenylethyl]-4,5-dihydro-1H-imidazole; 2-[2-(4-tolyl)-2-phenylethyl]-4,5-dihydro-1H-imidazole; 2-[2-(3-tolyl)-2-phenylethyl]-4,5-dihydro-1H-imidazole and 2-[2,2-bis(4-hydroxyphenyl)ethyl]-4,5-dihydro-1H-imidazole. It is proposed that the compounds have sedative action and that they potentiate narcosis and inhibit spontane motility.

JP 52-151165B (Daiichi Pharmaceutical Co.) describes 2-(2,2-diphenylethyl)-4,5-dihydro-1-methyl-1H-imidazole; 2-(2,2-diphenylethyl)-4,5-dihydro-4-methyl-1H-imidazole; 4-[2-(4,5-dihydro-1H-imidazol-2-yl)-1-phenylethyl]pyridine; 2-(2,2-diphenylethyl)-4,5-dihydro-1H-imidazole-1-carboxaldehyde; 1-acetyl-2-(2,2-diphenylethyl)-4,5-dihydro-1H-imidazole; 2-(2,2-diphenylethyl)-4,5-dihydro-1H-imidazole-1-carboxylic acid ethyl ester; 2-(2,2-diphenylethyl)-4,5-dihydro-1-hydroxymethyl-1H-imidazole and 2-(2,2-diphenylethyl)-1,4,5,6-tetrahydropyrimidin. It is proposed that the compounds have hypoglycaemic action and are useful in treating diabetes.

JP04-069385 (to Maruko Seiyaku) describes 2-(2-(4,5-dihydro-1H-imidazol-2-yl)-1-thiophene-2-yl-ethyl)pyridine. It is proposed that the compound has antidiabetic activity and is useful as a therapeutic or prophylactic agent for diabetes and its complications.

### BACKGROUND OF THE INVENTION

Diabetes is characterized by an impaired glucose metabolism manifesting itself among other things by an elevated blood glucose level in the diabetic patients. Underlying defects lead to a classification of diabetes into two major groups: type 1 diabetes, or insulin demanding diabetes mellitus (IDDM), which arises when patients lack β-cells producing insulin in their pancreatic glands, and type 2 diabetes, or non-insulin dependent diabetes mellitus (NIDDM), which occurs in patients with an impaired β-cell function besides a range of other abnormalities.

Type 1 diabetic patients are currently treated with insulin, while the majority of type 2 diabetic patients are treated either with compounds that stimulate β-cell function or with compounds that enhance the tissue sensitivity of the patients towards insulin.

Among the compounds applied for stimulation of the β-cell function, those acting on the ATP-dependent potassium channel of β-cells are most widely used in current therapy. Thus, sulphonylureas such as tolbutamide, glibenclamide, glipizide, and gliclazide are used extensively. Examples of other insulin secretagogues are repaglinide, nateglinide and KAD-1229. Examples of other types of compounds under investigation or in use in current therapy are alpha glucosidase inhibitors like miglitol and voglibose and insulin sensitizers like metformin, troglitazone, rosiglitazone and pioglitazone.

Even though sulphonylureas are widely used in the treatment of NIDDM this therapy is, in most instances, not satisfactory: In a large number of NIDDM patients sulphonylureas do not suffice to normalize blood sugar levels and the patients are, therefore, at high risk for acquiring diabetic complications. Also, many patients gradually lose the ability to respond to treatment with sulphonylureas and are thus gradually forced into insulin treatment. This shift of patients from oral hypoglycaemic compounds to insulin therapy is usually ascribed to exhaustion of the β-cells in NIDDM patients.

Another hormone, which plays an important role in the conversion of glucose, is glucagon One of the actions of glucagon is that it mediates the breakdown of glycogen, which results in a raise the blood glucose level. Therefore, hypersecretion of glucagon will aggravate the hyperglycaemia associated with type 2 diabetes. On the other hand, inhibition of glucagon secretion will help to norm alise the blood glucose level.

From the above it follows that it is highly desirable to find a pharmacologically active compound for the treatment of type 2 diabetes that will stimulate insulin release in a glucose-dependent manner and also inhibit glucagon secretion in a glucose-dependent manner.

Imidazoline compounds, (see e.g. U.S. Pat. No. 4,133,491 (Daiichi Seiyaku) and WO 91/00862 (Novo Nordisk A/S)) including several classical α-adrenoreceptor antagonists, have attracted considerable interest for more than a decade as possible therapeutic compounds in the treatment of type 2 diabetes. This is based on the ability of many imidazoline compounds to act as potent stimulators of insulin secretion. Good evidence exists that the insulinotropic actions of imidazolines result from inhibition of ATP-sensitive K⁺-channels (K_{ATP}-channels) in the pancreatic β-cell plasma membrane, causing membrane depolarisation, Ca²⁺ influx, and activation of the secretory machinery. In addition to these effects, imidazoline compounds may also stimulate insulin release by a direct interaction with the exocytotic machinery. The latter effect has been suggested to accelerate insulin release in a glucose-dependent manner.

Closure of the K_{ATP}-channels by pharmacologically active compounds will promote insulin secretion by initiating the same series of cellular events as glucose. Consequently, stimulation of insulin secretion by pharmacologically active compounds depends only to some extent on the ambient glucose concentration and is observed even in the absence of the sugar. On the contrary, compounds stimulating exocytosis by a K_{ATP}-channel independent mechanism, will only potentiate exocytosis when the normal stimulus-secretion pathway is stimulated by glucose or other nutrients which will produce the increase in [Ca²⁺], required for initiating the process of exocytosis. Such a compound will stimulate insulin secretion in a glucose-dependent manner and consequently minimise the risk for hypoglycaemia.

In type 2 diabetes, the blood glucose concentration is chronically elevated partly due to insufficient release of insulin from the β-cells. Sulphonylureas have been used clinically for several years to stimulate insulin secretion in patients with type 2 diabetes. They act by inhibiting the K_{ATP}-channels independently of the glucose metabolism in the pancreatic β-cell. Although sulphonylureas remain the best documented and most commonly used class of oral antidiabetic compounds, they do not restore the physiological pattern of insulin secretion induced by glucose and other nutrients. Consequently, the use of sulphonylureas in the treatment of type 2 diabetes can potentially result in hypoglycaemic episodes and excessive stimulation of Ca²⁺ influx, which may induce apoptosis and a reduction in the β-cell mass. The latter mechanism might contribute to the so-called ‘secondary failure’ that occurs in the majority of patients with type 2 diabetes, when sulphonylureas eventually fail to normalise blood glucose.

The pancreatic β-cell contains approximately 13000 insulin-containing granules. However, only around 100 insulin granules fuse with the plasma membrane and release their contents when intracellular Ca²⁺ is increased. The remaining granules must undergo a maturation process before they can be released upon elevation of intracellular Ca²⁺. This maturation process is referred to as priming of the insulin granules for release. We speculate that imidazoline compounds that selectively stimulate priming of insulin granules in the pancreatic β-cell, will only increase the number of granules that are released in response to a blood-glucose increase. Such compounds will counteract the insufficient or sluggish insulin release seen in diabetic patients, without the risk for excessive pancreatic β-cell stimulation or risk for hypoglycaemia.

Imidazoline compounds not only stimulate insulin release but also inhibit glucagon secretion. This is a particularly important feature since hypersecretion of glucagon aggravates the hyperglycaemia associated with type 2 diabetes. This suggests that inhibition of glucagon secretion will help to normalise the blood glucose concentration.

### SUMMARY OF THE INVENTION

The compounds of formula (I) may exist as geometric and optical isomers and all isomers and mixtures thereof are included herein. Isomers may be separated by means of standard methods such as chromatographic techniques or fractional crystallization of suitable salts.

Preferably, when such forms exist, the compounds of formula (I) are used in the form of the individual geometric or optical isomers.

The compounds according to the invention may optionally be provided in the form of pharmaceutically acceptable acid addition salts. Examples of such salts include inorganic and organic acid addition salts such as hydrochloride, hydrobromide, sulphate, phosphate, acetate, fumarate, maleate, citrate, lactate, tartrate, oxalate or similar pharmaceutically acceptable inorganic or organic acid addition salts, and include the pharmaceutically acceptable salts listed in Journal of Pharmaceutical Science, 66, 2 (1977) which are hereby incorporated by reference.

Compounds of the invention which have a carboxylic acid group may exist in the form of salts with pharmaceutically acceptable cations e.g. sodium ions, calcium ions and optionally substituted ammonium ions.

Definitions

The term “halogen” means fluorine, chlorine, bromine or iodine.

The term “alkyl” includes C₁₋₆ straight chain saturated aliphatic hydrocarbon groups, methylene and C₂₋₆ unsaturated aliphatic hydrocarbon groups, C₃₋₆ branched saturated and C₂₋₆ unsaturated aliphatic hydrocarbon groups, C₃₋₆ cyclic saturated and C₆₋₆ unsaturated aliphatic hydrocarbon groups, and C₁₋₆ straight chain or branched saturated and C₂₋₆ straight chain or branched unsaturated aliphatic hydrocarbon groups substituted with C₃₋₆ cyclic saturated and unsaturated aliphatic hydrocarbon groups having the specified number of carbon atoms. For example, this definition shall include but is not limited to methyl (Me), ethyl (Et), propyl (Pr), butyl (Bu), pentyl, hexyl, ethenyl, propenyl, butenyl, pentenyl, hexenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cyclopentenyl, cyclohexenyl, methylcyclopropyl, ethylcyclohexenyl, butenylcyclopentyl, ethynyl, propynyl and butynyl.

The term “substituted alkyl” represents an alkyl group as defined above wherein the substitutents are independently selected from halo, cyano, nitro, trihalomethyl, carbamoyl, hydroxy, oxo, —COOR²⁰, —C(O)—NR²¹R²², C₁₋₆alkyloxy-, aryloxy-, arylC₁₋₆alkyloxy-, C₁₋₆alkylthio-, arylthio-, arylC₁₋₆alkylthio-, —NR²³R²⁴, C₁₋₆alkyl-C(O)—, arylC₁₋₆alkyl-C(O)—, R²⁵—C(O)—N(R²⁶)—, morpholinyl, or piperazinyl; wherein R²⁰ is H, C₁₋₆alkyl or arylC₁₋₆alkyl; R²¹ and R²² are independently hydrogen, C₁₋₆alkyl or arylC₁₋₆alkyl; R²³ and R²⁴ are independently hydrogen, C₁₋₆alkyl, arylC₁₋₆alkyl, COOR²⁰, or the groups R²³ and R²⁴ may together with the nitrogen atom to which they are attached form a saturated, partially saturated or aromatic monocyclic or bicyclic ring system containing 3 to 14 carbon atoms and 0 to 3 additional heteroatoms independently selected from nitrogen, oxygen or sulfur, the ring system optionally being substituted with at least one C₁₋₆alkyl, aryl, arylC₁₋₆alkyl or oxo substituent; R²⁵ is C₁₋₆alkyl, aryl, arylC₁-6alkyl or —NR²³R²⁴; wherein R²³ and R²⁴ are defined as above; R²⁶ is C₁₋₆alkyl, aryl or arylC₁₋₆alkyl.

The term “saturated, partially saturated or aromatic monocyclic, bicyclic or tricyclic ring system” comprises but is not limited to aziridinyl, pyrrolyl, pyrrolinyl, pyrrolidinyl, imidazolyl, 2-imidazolinyl, imidazolidinyl, pyrazolyl, 2-pyrazolinyl, 1,2,3-triazolyl, 1,2,4-triazolyl, tetrazolyl, morpholinyl, piperidinyl, thiomorpholinyl, piperazinyl, indolyl, isoindolyl, 1,2,3,4-tetrahydroquinolinyl, 1,2,3,4-tetrahydro-isoquinolinyl, 1,2,3,4-tetrahydro-quinoxalinyl, indolinyl, indazolyl, benzimidazolyl, benzotriazolyl, purinyl, carbazolyl, acridinyl, phenothiazinyl, phenoxazinyl, iminodibenzyl and iminostilbenyl.

The term “arylalkyl” (e.g. benzyl, phenylethyl) represents an “aryl” group as defined below attached through an alkyl having the indicated number of carbon atoms or substituted alkyl group as defined above.

The term “alkyloxy” (e.g. methoxy, ethoxy, propoxy, isopropoxy, butoxy, pentoxy) as used herein, alone or in combination, refers to a straight or branched monovalent substituent comprising an alkyl group as defined above linked through an ether oxygen having its free valence bond from the ether oxygen and having from 1 to 6 carbon atoms.

The term “aryloxy” (e.g. phenoxy, naphthyloxy and the like) represents an “aryl” group as defined below attached through an oxygen bridge.

The term “arylalkyloxy” (e.g. phenethyloxy, naphthylmethyloxy and the like) represents an “arylalkyl” group as defined above attached through an oxygen bridge.

The term “alkyloxyalkyl” represents an “alkyloxy” group attached through an alkyl group as defined above having the indicated num ber of carbon atoms.

The term “arylalkyloxy” (e.g. phenethyloxy, naphthylmethyloxy and the like) represents an “arylalkyl” group as defined below attached through an oxygen bridge.

The term “arylalkyloxyalkyl” represents an “arylalkyloxy” group as defined above attached through an “alkyl” group defined above having the indicated number of carbon atoms.

The term “alkylthio” (e.g. methylthio, ethylthio, propylthio, cyclohexenylthio and the like) represents an “alkyl” group as defined above having the indicated number of carbon atoms attached through a sulfur bridge.

The term “arylalkylthio” (e.g. phenylmethylthio, phenylethylthio, and the like) represents an “arylalkyl” group as defined above having the indicated number of carbon atoms attached through a sulfur bridge.

The term “arylthio” (e.g. phenylthio, naphthylthio and the like) represents an “aryl” group as defined below attached through a sulfur bridge.

The term “aryl” represents an unsubstituted, mono-, di- or trisubstituted monocyclic, polycyclic, biaryl and heterocyclic aromatic groups covalently attached at any ring position capable of forming a stable covalent bond, certain preferred points of attachment being apparent to those skilled in the art (e.g., 3-indolyl, 4(5)-imidazolyl).

The definition of aryl includes but is not limited to phenyl, biphenyl, indenyl, fluorenyl, naphthyl (1-naphthyl, 2-naphthyl), pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), isoxazolyl (3-isoxazolyl, 4-isoxazolyl, 5-isoxazolyl), thiazolyl (2-thiazolyl, 4-thiazolyl, 5-thiazolyl), thienyl (2-thienyl, 3-thienyl, 4-thienyl, 5-thienyl), furanyl (2-furanyl, 3-furanyl, 4-furanyl, 5-furanyl), pyridyl (2-pyridyl, 3-pyridyl, 4-pyridyl, 5-pyridyl), 5-tetrazolyl, pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl).

The term “substituted aryl” represents an aryl group as defined above where the substituents are independently selected from the group consisting of halogen, nitro, cyano, trihalogenomethyl, C₁₋₆alkyl, aryl, arylC₁₋₆alkyl, hydroxy, —COOR²⁰, —C(O)—NR²¹R²², —SO—NR²¹R²², —SO₂—NR²¹R²², C₁₋₆alkyloxy-, C₁₋₆alkyloxyC₁₋₆alkyl, aryloxy-, arylC₁₋₆alkyloxy-, arylC₁₋₆alkyloxyC₁₋₆alkyl, C₁₋₆alkylthio-, arylthio-, arylC₁₋₆alkylthio-, —NR²³R²⁴, C₁₋₆alkyl-C(O)—, arylC₁₋₆alkyl-C(O)—, or R²⁵—C(O)—N(R²⁶)—; wherein R²⁰, R²¹, R²², R²³, R²⁴, R²⁵ and R²⁶ are defined as above and the alkyl and aryl groups are optionally substituted as defined above.

The compounds according to the present invention have interesting pharmacological properties. In particular, the compounds stimulate insulin release and inhibit glucagon secretion in a glucose-dependent manner. Therefore, the compounds can be used as medicaments, either alone or in combination with another pharmacologically active compound, optionally together with a suitable carrier. In particular, the compounds can be used, either alone or in combination with another pharmacologically active compound, optionally together with a suitable carrier, as a medicament in the treatment of type 2 diabetes. Furthermore, the compounds of the invention can be used, either alone or in combination with another pharmacologically active compound and optionally together with a suitable carrier, in the manufacture of a medicament, in particular in the manufacture of a medicament for the treatment of type 2 diabetes. Also, within the scope of the invention is a method for the treatment of type 2 diabetes.

### BRIEF DESCRIPTION OF THE DRAWING

The present invention is described with reference to the appended drawing wherein: FIGS. 1 and 2 show the effects of the title compound of Example 2, in the following designated “compound (Ex 2)” on Ca²⁺-evoked exocytosis in mouse pancreatic β-cells. Thus, FIG. 1 shows the increases in cell capacitance (reflecting exocytosis) elicited by intracellular infusion with a Ca²⁺-EGTA buffer (EGTA=ethylene glycol-bis(β-aminoethyl ether) N,N,N′,N′-tetraacetic acid) with a free Ca²⁺ concentration of 0.2 μM in the absence (control) and presence of 100 μM compound (Ex 2) in the pipette solution observed during the first two minutes after establishment of the standard whole-cell configuration. Throughout the recording, the cell was voltage-clamped at −70 mV in order to avoid activation of the voltage-dependent Ca²⁺ channels that would otherwise interfere with the measurement. The recordings were obtained from different cells.

FIG. 2 shows a histogram depicting average rates of increase in cell capacitance (ΔCₘ/Δt, for a definition, see data analysis section) in the absence and presence of 100 μM compound (Ex 2) measured over the first 60 seconds following establishment of the whole-cell configuration. Data are mean values ±S.E.M. of 13 (control) and 6 (compound (Ex 2)) different cells. *P<0.025.

FIG. 3 shows that compound (Ex 2) fails to reduce whole-cell ATP-sensitive K⁺ currents in mouse β-cells. The whole-cell currents were measured in response to 10 mV de- and re-polarising voltage pulses from a holding potential of −70 mV. Once steady state had been attained (2-4 min) 100 μM compound (Ex 2) was applied and the current responses were measured for another 5-10 min. In a series of six different experiments, compound (Ex 2) at a concentration of 100 μM did not affect the whole-cell K_{ATP}-current (90+4% of prestimulatory level).

FIG. 4 shows that compound (Ex 2) only stimulates insulin release from intact mouse islets in the presence of a stimulatory glucose concentration. Batches of 10 islets, cultured overnight in RPMI-1640 medium, were exposed to the indicated concentrations of glucose alone or in the presence of 100 μM of compound (Ex 2). Following 60 min incubation, the supernatant was aspired and analysed for insulin content. Data are mean±S.E.M of eight measurements. *P<0.05.

FIG. 5 shows the effects of compound (Ex 2) on Ca²⁺-dependent exocytosis in single rat pancreatic α-cells. In these experiments exocytosis was stimulated with a maximal effective Ca²⁺ concentration of 2 μM, which under control conditions produced a large increase in exocytosis. However, inclusion of compound (Ex 2) in a concentration of 0.1 mM produced a strong inhibition of the exocytotic response.

FIG. 6 shows a histogram depicting average rates of increase in cell capacitance (ΔCₘ/Δt) in the absence and presence of 100 μM compound (Ex 2) measured over the first 60 seconds following establishment of the whole-cell configuration. Data are mean values ±S.E.M. of 5 different cells. *P<0.01.

FIG. 7 shows the glucose dependent insulin release by compound (Ex 2) in isolated perfused rat pancreas.

### DETAILED DESCRIPTION OF THE INVENTION

In one aspect, the present invention relates to compounds of the general formula (I):

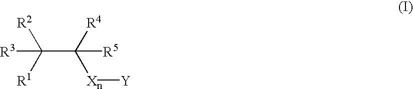

wherein Y is selected from the following ring systems:

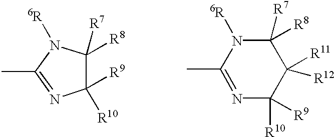

R¹ and R² are independently phenyl, naphthyl, thienyl, thiazolyl, imidazolyl, pyrazolyl, triazolyl, pyridyl, pyrimidyl, pyridazinyl, pyrazinyl, quinolyl, 3,4-dihydroquinolyl, 5,6,7,8-tetrahydroquinolyl, isoquinolyl, 3,4-dihydroisoquinolyl, 5,6,7,8-tetrahydroisoquinolyl, indolyl, benzo[b]thienyl, benzimidazolyl, 5,6,7,8-tetrahydroimidazo[1,5-a]pyridyl or benzthiazolyl each of which is optionally substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, -arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;

R³ and R⁵ are independently hydrogen, C₁₋₆alkyl or halogen or when taken together R³ and R⁵ form an additional bond between the carbon atoms to which they are attached;

R⁴ is hydrogen, C₁₋₆alkyl or halogen;

R⁶ is hydrogen, C₁₋₆alkyl, arylC₁₋₆alkyl, C₁₋₆alkyl-C(O)— or arylC₁₋₆alkyl-C(O)— wherein the alkyl groups are optionally substituted with halogen, methoxy or ethoxy and the aryl groups are optionally substituted with halogen, methyl, ethyl, methoxy or ethoxy;

R⁷, R⁸, R⁹, R¹⁰, R¹¹, and R¹² are independently hydrogen, C₁₋₆alkyl, aryl or arylC₁₋₆alkyl;

wherein the alkyl groups are optionally substituted with halogen, methoxy or ethoxy and the aryl groups are optionally substituted with halogen, methyl, ethyl, methoxy or ethoxy;

X is —CR¹⁸R¹⁹— wherein R¹⁸ and R¹⁹ are independently hydrogen or C₁₋₆alkyl;

n is 0, 1 or 2; and any stereoisomers and geometrical isomers and salts thereof with the proviso that the compound of formula (I) is not one of the following compounds:

2-(2,2-diphenylethyl)-4,5-dihydro-1H-imidazole;

2-[2-phenyl-2-(2-pyridyl)]ethyl-2-imidazoline;

2-[2-(4-chlorophenyl)-2-(2-pyridyl)]ethyl-2-imidazoline;

2-[2-(indol-3-yl)-2-phenyl]ethyl-2-imidazoline;

2-[2-(2-methylindol-3-yl)-2-phenyl]ethyl-2-imidazoline;

2-[2-(indol-3-yl)-2-(3-methoyxphenyl)]ethyl-2-imidazoline;

2-[2-(2-methylindol-3-yl)-2-(3-methoxyphenyl)]ethyl-2-imidazoline;

2-[2-(5-chloro-2-methylindol-3-yl)-2-(3-methoxyphenyl)]ethyl-2-imidazoline;

2-[2-(4-chlorophenyl)-2-phenylethyl]-4,5-dihydro-1H-imidazole;

2-[2-(4-tolyl)-2-phenylethyl]-4,5-dihydro-1H-imidazole;

2-[2-(3-tolyl)-2-phenylethyl]-4,5-dihydro-1H-imidazole;

2-[2,2-bis(4-hydroxyphenyl)ethyl]-4,5-dihydro-1H-imidazole;

2-(2, 2-diphenylethyl)-4,5-dihydro-1-methy-1H-imidazole;

2-(2, 2-diphenylethyl)-4,5-dihydro-4-methyl-1H-imidazole;

4-[2-(4,5-dihydro-1H-imidazol-2-yl)-1-phenylethyl]pyridine;

2-(2, 2-diphenylethyl)-4,5-dihydro-1H-imidazole-1-carboxaldehyde;

1-acetyl-2-(2,2-diphenylethyl)-4,5-dihydro-1H-imidazole;

2-(2, 2-diphenylethyl)-4,5-dihydro-1H-imidazole-1-carboxylic acid ethyl ester;

2-(2,2-diphenylethyl)-4,5-dihydro-1-hydroxymethyl-1H-imidazole;

2-(2, 2-diphenylethyl)-1,4,5,6-tetrahydropyrimidine; and

2-(2-(4,5-dihydro-1H-imidazol-2-yl)-1-thiophene-2-yl-ethyl)pyridine.

In another aspect, the present invention relates to compounds of the general formula (I):

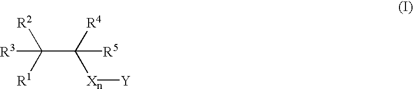

wherein Y is selected from the following ring systems:

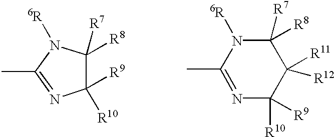

R¹ and R² are independently naphthyl, 3-thienyl, thiazolyl, imidazolyl, pyrazolyl, triazolyl, 3-pyridyl, pyrimidyl, pyridazinyl, pyrazinyl, quinolyl, 3,4-dihydroquinolyl, 5,6,7,8-tetrahydroquinolyl, isoquinolyl, 3,4-dihydroisoquin olyl, 5,6,7,8-tetrahydroisoquinolyl, 4-indolyl, 7-indolyl, benzo[b]thienyl, benzimidazolyl, 5,6,7,8-tetrahydroimidazo[1,5-a]pyridyl or benzthiazolyl each of which is optionally substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;

with the further option that R¹ or R² or both R¹ and R² are selected from the group consisting of 2-thienyl, 2-pyridyl and 4-pyridyl substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;

with the still further option that R¹ or R² or both R¹ and R² are selected from the group consisting of phenyl substituted with C₂₋₆alkyl, hydroxy, C₁₋₆alkoxy, cyano, trifluoromethoxy, fluorine, bromine, iodine, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;

R³ and R⁵ are independently hydrogen, C₁₋₆alkyl or halogen or when taken together R³ and R⁵ form an additional bond between the carbon atoms to which they are attached;

R⁴is hydrogen, C₁₋₆alkyl or halogen;

R⁶ is hydrogen, C₁₋₆alkyl, arylC₁₋₆alkyl, C₁₋₆alkyl-C(O)— or arylC₁₋₆alkyl-C(O)— wherein the alkyl groups are optionally substituted with halogen, methoxy or ethoxy and the aryl groups are optionally substituted with halogen, methyl, ethyl, methoxy or ethoxy;

R⁷, R⁸, R⁹, R¹⁰, R¹¹, and R¹² are independently hydrogen, C₁₋₆alkyl, aryl or arylC₁₋₆alkyl;

wherein the alkyl groups are optionally substituted with halogen, methoxy or ethoxy and the aryl groups are optionally substituted with halogen, methyl, ethyl, methoxy or ethoxy;

X is —CR¹⁸R¹⁹— wherein R¹⁸ and R¹⁹ are independently hydrogen or C₁₋₆alkyl;

n is 0, 1 or 2; and any stereoisomers and geometrical isomers and salts thereof.

In one embodiment, R¹ and R² are identical groups. In another embodiment, R¹ and R² are differentfrom each other. In a further embodiment, one of R¹ and R² is an optionally substituted carbocyclic aryl group and the other one is an optionally substituted heterocyclic aryl group.

In a further embodiment of the invention, R³ and R⁵ are hydrogen. In a further embodiment of the invention, R³, R⁴ and R⁵ are hydrogen.

In a further embodiment of the invention, R3 and R⁵ together form an additional bond between the carbon atoms to which thet are attached.

In a further embodiment of the invention, n is 0. In a further embodiment of the invention, n is 1. In a further embodiment of the invention, n is 2.

In a further embodiment of the invention, Y is

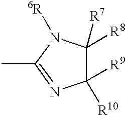

and R⁶, R⁷, R⁸, R⁹ and R¹⁰ are as defined above. With this definition of Y, R⁶, R⁷, R⁸, R⁹ and R¹⁰ can all be hydrogen, or one of R⁶, R⁷, R⁸, R⁹ and R¹⁰ can be C₁₋₆alkyl while the remaining groups are hydrogen. Thus, for example, one of R⁶, R⁷, R⁸, R⁹ and R¹⁰ can be methyl while the remaining groups are hydrogen. As a further option, one of R⁶, R⁷, R⁸, R⁹ and R¹⁰ can be arylC₁₋₆alkyl while the remaining groups are hydrogen. Thus, for example, one of R⁶, R⁷, R⁸, R⁹ and R¹⁰ can be benzyl while the remaining groups are hydrogen. With this definition of Y, R¹ can be a heteroaryl group, in particular a pyridyl group, and R² can be 3-methoxyphenyl and n can be 0. Also, R¹ can be 2-thienyl or 3-thienyl while R² is a substituted carbocyclic or heterocyclic group. Furthermore, R¹ can be 2-chlorophenyl or 3-chlorophenyl while R²is an optionally substituted carbocyclic or heterocyclic group or R¹ can be 4-chlorophenyl R² is a substituted carbocyclic or heterocyclic group.

In a further embodiment of the invention, Y is

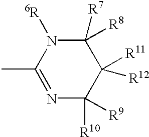

and R⁶, R⁷, R⁸, R⁹, R¹⁰, R¹¹ and R¹² are as defined above. With this definition of Y, R⁶, R⁷, R⁸, R⁹, R¹⁰, R¹¹ and R¹² can all be hydrogen, or one of R⁶, R⁷, R⁸, R⁹, R¹⁰, R¹¹ and R¹² can be C₁₋₆alkyl while the remaining groups are hydrogen. Thus, for example, one of R⁶, R⁷, R⁸, R⁹, R¹⁰, R¹¹ and R¹² can be methyl while the remaining groups are hydrogen. As a further option, one of R⁶, R⁷, R⁸, R⁹, R¹⁰, R¹¹ and R¹² can be arylC₁₋₆alkyl while the remaining groups are hydrogen. Thus, for example, one of R⁶, R⁷, R⁸, R⁹, R¹⁰, R¹¹ and R¹² can be benzyl while the remaining groups are hydrogen.

Within the scope of the present invention, compounds of formula (I) may be prepared in the form of acid addition salts, in particular pharmaceutically acceptable acid addition salts, with inorganic or organic acids. Examples of such salts include inorganic acid addition salts such as salts of hydrochloric, hydrobromic, sulphuric and phosphoric acids and the like. Suitable organic acid addition salts include salts of formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Further examples of pharmaceutically acceptable inorganic or organic acid addition salts include the pharmaceutically acceptable salts listed in J. Pharm. Sci., 66, 2 (1977), which are known to the skilled artisan.

Also intended as pharmaceutically acceptable acid addition salts are hydrates formed by the acid addition salts.

The acid addition salts may be obtained as the direct products of compound synthesis. In the alternative, a compound of formula (I) in the form of the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

In one embodiment, the invention relates to compounds of formula (I) and salts thereof with pharmacologically acceptable inorganic and organic acids.

Salts of compounds of formula (I) with inorganic or organic acids, which are not necessarily pharmacologically acceptable, are also within the scope of the present invention. Such salts may be useful as intermediates during the production of compounds of formula (I) and pharmacologically acceptable salts thereof with inorganic or organic acids, e.g. in a purification step or in a resolution step. Eventually, such salts will of course have to be converted to the free base or to a pharmacologically acceptable acid addition salt.

In another embodiment, the invention relates to compounds of formula (I) or a salt thereof, which is essentially optically pure.

In a further embodiment, the invention relates to compounds of formula (I) or a salt thereof with an optical purity of at least ee=98%

In a further embodiment, the invention relates to compounds of formula (I) or a salt thereof with an optical purity of at least ee=95%

The present invention also relates to a pharmaceutical composition comprising a compound of formula (I) or a salt thereof with a pharmacologically acceptable inorganic or organic acid together with a pharmaceutically acceptable carrier.

In a further embodiment, the invention relates to a pharmaceutical composition comprising a compound of formula (I) or a salt thereof with a pharmacologically acceptable inorganic or organic acid together with a further pharmacologically active compound, useful in the treatment of type 2 diabetes, and a pharmaceutically acceptable carrier.

In a further aspect of the invention, a compound of formula (I) may be administered in combination with a further pharmacologically active compound, either contained in the same dosage unit or comprised in a pharmaceutical kit. Such further pharmacologically active substances may be selected among glucagon-like peptide 1 (GLP-1) and analogues and derivatives thereof, e.g. as described in WO 98/08871 (Novo Nordisk A/S) which is incorporated herein in its entirety by reference. Further options may be selected from the group consisting of alpha glucosidase inhibitors like miglitol and voglibose, and from the group consisting of hepatic enzyme inhibitor of the kind described in WO 95/24391 (Novo Nordisk A/S).

In a further aspect, the present invention relates to a method of treating type 2 diabetes which method comprises administering an effective amount of a compound of the invention to a patient in need of such a treatment, optionally together with a pharmaceutically acceptable carrier.

In a still further aspect, the present invention relates to the use of a compound of formula (I) or a salt thereof with a pharmaceutically acceptable inorganic or organic acid for the preparation of a medicament for the treatment of type 2 diabetes.

In a still further aspect, the present invention relates to a method of treating type 2 diabetes in a patient in need of such a treatment, comprising administering to the patient a therapeutically effective amount of a composition according to the invention, optionally together with a pharmaceutically acceptable carrier.

Examples of compounds according to the invention are given in the following list:

2-(2-(4,5-Dihydro-1H-imidazol-2-yl)-1-(2-methoxyphenyl)ethyl)pyridine;

2-(2-(4,5-Dihydro-1H-imidazol-2-yl)-1-(2-pyridyl)ethyl)pyridine;

3-(2-(4,5-Dihydro-1H-imidazol-2-yl)-1-phenylethyl)-5,6,7,8-tetrahydroimidazo[1,5-a]pyridine;

2-(2-(4,5-Dihydro-1H-imidazol-2-yl)-1-naphthalen-1-ylethyl)pyridine;

1-(2-(4,5-Dihydro-1H-imidazol-2-yl)-1-pyridine-2-ylethyl)isoquinoline;

2-(2-(4,5-Dihydro-1H-imidazol-2-yl)-1-pyridine-2-ylethyl)quinoline;

2-(2-(4,5-Dihydro-1H-imidazol-2-yl)-1-phenylethyl)-1-methyl-1H-imidazole;

2-(2-(4,5-Dihydro-1H-imidazol-2-yl)-1-(2-methoxymethylphenyl)ethyl)pyridine;

2-(2-(4,5-Dihydro-1H-imidazol-2-yl)-1-(2-et hylphenyl)ethyl)pyridine;

2-(2-(4,5-Dihydro-1H-imidazol-2-yl)-1-(3-methoxyphenyl)ethyl)pyridine;

5-Bromo-2-(2-(4,5-dihydro-1H-imidazol-2-yl)-1-phenylethyl)-1-methyl-1H-imidazole;

2-(2-(4,5-Dihydro-1H-imidazol-2-yl)-1-(2-methoxy-5-methylphenyl)ethyl)pyridine;

2-(2-(4,5-Dihydro-1H-imidazol-2-yl)-1-thiazol-2-ylethyl)pyridine;

2-(2-(4,5-Dihydro-1H-imidazol-2-yl)-1-(2,5-dimethoxyphenyl)ethyl)pyridine;

2-(2-(4,5-Dihydro-1H-imidazol-2-yl)-1-(3,5-dimethoxyphenyl)ethyl)pyridine;

3-(2-(4,5-Dihydro-1H-imidazol-2-yl)-1-thiophene-2-ylethyl)pyridine;

4-(2-(4,5-Dihydro-1H-imidazol-2-yl)-1-thiophene-3-ylethyl)pyridine;

4-(2-(4,5-Dihydro-1H-imidazol-2-yl)-1-thiophene-2-ylethyl)pyridine;

2-(2-(4,5-Dihydro-1H-imidazol-2-yl)-1-(3,4-dimethoxyphenyl)ethyl)pyridine;

2-(2-(4,5-Dihydro-1H-imidazol-2-yl)-1-(3-ethoxyphenyl)ethyl)pyridine;

3-Chloro-2-(2-(4,5-dihydro-1H-imidazol-2-yl)-1-(4-methoxyphenyl)ethyl)-5-trifluoromethylpyridine;

1-{2-[2-(3-Methoxy phenyl)-2-pyridine-2-ylethyl]-4,5-dihydroimidazol-1-yl}ethanone;

{2-[2-(3-Methoxy phenyl)-2-pyridine-2-ylethyl]-4,5-dihydroimidazol-1-yl}phenylmethanone;

2-[1-Benzo[1,3]dioxol-5-yl-2-(4,5-dihydro-1H-imidazol-2-yl)ethyl]pyridine;

2-[2-(4,5-Dihydro-1H-imidazol-2-yl)-1-(3-methoxyphenyl)ethyl]quinoline;

1-[2-(4,5-Dihydro-1H-imidazol-2-yl)-1-(3-methoxyphenyl)ethyl]isoquinoline;

2-[1-(3-Chlorophenyl)-2-(4,5-dihydro-1H-imidazol-2-yl)ethyl]pyridine;

1-[2-(4,5-Dihydro-1H-imidazol-2-yl)-1-(3-methoxyphenyl)vinyl]isoquinoline;

2-[2-(4,5-Dihydro-1H-imidazol-2-yl)-1-(3-methoxyphenyl)vinyl]quinoline;

2-[1-Benzo[1,3]dioxol-5-yl-2-(4,5-dihydro-1H-imidazol-2-yl)vinyl]pyridine; and

3-[2-(4,5-Dihydro-1H-imidazol-2-yl)-1-thiophene-3-ylvinyl]pyridine.

and pharmaceutically acceptable salts thereof.

Compounds which stimulate insulin secretion in a glucose-dependent manner while at the same time suppressing glucagon release can be identified by screening the effects of the compounds and their enantiomers on Ca²⁺-dependent exocytosis, insulin release, K_{ATP}-channel activity and glucagon secretion in either single pancreatic β-cells or in intact islets of Langerhans. For a more detailed description of the test system, see the experimentral section.

The compounds of the present invention are useful in the treatment of diabetes, in particular Type 2 diabetes. Thus, they are useful for normalising hyperglycaemic and for preventing or alleviating diabetic complications, including late complications, such as retinopathy, neuropathy, nephropathy, and micro- and macroangiopathy; hypercholesterolemia, hypertension, hyperinsulinemia, hyperlipidemia, atherosclerosis or ischemia or treatment or prophylaxis of obesity and appetite regulation.

Pharmaceutical Compositions

The present invention includes within its scope pharmaceutical compositions comprising, as an active ingredient, a compound of formula (I) or a salt thereof with a pharmaceutically acceptable inorganic or organic acid together with a pharmaceutically acceptable carrier or diluent.

Optionally, the pharmaceutical composition of the invention may comprise a compound of formula (I) or a salt thereof with a pharmaceutically acceptable inorganic or organic acid combined with one or more compounds having a different pharmacological action, e.g. a further antidiabetic compound or other pharmacologically active material.

Pharmaceutical compositions containing a compound of formula (I) or a salt thereof with a pharmaceutically acceptable inorganic or organic acid may be prepared by conventional techniques, e.g. as described in Remington: The Science and Practise of Pharmacy, 19^{th} Ed., 1995. The compositions may appear in conventional forms, for example capsules, tablets, aerosols, solutions, suspensions or topical applications, e.g. patches.

Typical compositions include a compound of formula (I), or a salt thereof with a pharmaceutically acceptable inorganic or organic acid, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, agents for influencing the osmotic pressure of a solution, buffers and/or colouring substances and the like, which do not deleteriously react with the active compounds.

The route of administration may be any route, which effectively transports the active compound to the appropriate or desired site of action, such as oral, nasal, pulmonary, transdermal (e.g. via a patch) or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of formula (I) or a salt thereof with a pharmaceutically acceptable inorganic or organic acid dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch and/or potato starch.

A typical tablet, which may be prepared by conventional tabletting techniques, may contain:

| Core: | | |
|---|---|---|
| Active compound (as free compound or salt thereof) | 2 | mg |
| Colloidal silicon dioxide (Aerosil) | 1.5 | mg |
| Cellulose, microcryst. (Avicel) | 70 | mg |
| Modified cellulose gum (Ac-Di-Sol) | 7.5 | mg |
| Magnesium stearate | Ad. 90 | mg |
| Coating: | | |
| HPMC approx. | 9 | mg |
| *Mywacett 9-40 T approx. | 0.9 | mg |

*An acylated monoglyceride used as plasticizer for film coating.

The compounds of the invention may be administered to a mammal, especially a human in need of such treatment, prevention, elimination, alleviation or amelioration of the various diseases as mentioned herein, e.g. hyperglycemia, hypercholesterolem ia, hypertension, hyperinsulinemia, hyperlipidemia, or obesity, and especially diabetes. Such mammals include also animals, both domestic animals, e.g. household pets, and non-domestic animals such as wildlife.

The compounds of the invention are effective over a wide dosage range. For example, in the treatment of adult humans, dosages from about 0.05 to about 1000 mg, preferably from about 0.1 to about 500 mg, per day may be used. A most preferable dosage is about 0.5 mg to about 250 mg per day. In choosing a regimen for patients it may frequently be necessary to begin with a higher dosage and when the condition is under control to reduce the dosage. The exact dosage will depend upon the mode of administration, on the therapy desired, form in which administered, the subject to be treated and the body weight of the subject to be treated, and the preference and experience of the physician or veterinarian in charge.

Generally, the compounds of the present invention are dispensed in unit dosage form comprising from about 0.05 to about 500 mg of active ingredient together with a pharmaceutically acceptable carrier per unit dosage.

Usually, dosage forms suitable for oral, nasal, pulmonal or transdermal administration comprise from about 0.05 mg to about 500 mg, preferably from about 0.5 mg to about 250 mg of the compounds of formula I, Ia, or Ib admixed with a pharmaceutically acceptable carrier or diluent.

The invention also encompasses prodrugs of a compound of the invention, which on administration undergo chemical conversion by metabolic processes before becoming active pharmacological substances. In general, such prodrugs will be functional derivatives of a compound af the invention which are readily convertible in vivo into a compound af the invention. Conventional procedures for the selection and preparation of suitable prodrug derivatives are described, for example, in “Design of Prodrugs”, ed. H. Bundgaard, Elsevier, 1985.

The invention also encompasses active metabolites of a compound of the invention.

In one aspect of the invention, a compound of the invention may be administered in combination with further pharmacologically active substances eg antiobesity agents or appetite regulating agents.

Such further pharmacologically active agents may be selected from the group consisting of CART agonists, NPY antagonists, MC4 agonists, orexin antagonists, TNF agonists, CRF agonists, CRF BP antagonists, urocortin agonists, β3 agonists, MSH (melanocyte-stimulating hormone) agonists, MCH (melanocyte-concentrating hormone) antagonists, CCK agonists, serotonin re-uptake inhibitors, mixed serotonin and noradrenergic compounds, 5HT agonists, bombesin agonists, galanin antagonists, growth hormone, growth hormone releasing compounds, TRH agonists, uncoupling protein 2 or 3 modulators, GLP-1, leptin agonists, DA agonists (bromocriptin, doprexin), lipase/amylase inhibitors PPAR modulators, RXR modulators or TR β agonists.

In a preferred embodiment of the invention the antiobesity agent is leptin.

In another preferred embodiment the antiobesity agent is dexamphetamine or amphetamine.

In another preferred embodiment the antiobesity agent is dexfenfluramine.

In still another preferred embodiment the antiobesity agent is sibutramine.

In a further preferred embodiment the antiobesity agent is orlistat.

In a further preferred embodiment the antiobesity agent is mazindol or phentermine.

In another aspect of the invention, a compound of the invention may be administered in combination with a lipid lowering agent.

A compound of the invention may also be administered in combination with an antidiabetic or other pharmacologically active material, including compounds for the treatment and/or prophylaxis of insulin resistance and diseases, wherein insulin resistance is the pathophysiological mechanism. Suitable antidiabetics comprise insulin, insulin analogues, GLP-1 derivatives such as those disclosed in WO 98/08871 to Novo Nordisk A/S, which is incorporated herein by reference as well as orally active hypoglycaemic agents, not modulating the K_{ATP} channel in the β-cell.

The orally active hypoglycaemic agents preferably comprise biguanides, in particular metformin, oxadiazolidinediones, thiazolidinediones, a-glucosidase inhibitors, glucagon antagonists, GLP-1 agonists, insulin sensitizers, hepatic enzyme inhibitors, glucose uptake modulators, compounds modifying the lipid metabolism, compounds lowering food intake, PPAR and RXR agonists.

In a preferred embodiment of the invention a compound of the invention is administered in combination with insulin or an insulin analogue.

In another preferred embodiment a compound of the invention is administered in combination with a biguanide like metformin.

In still another preferred em bodiment a compound af the invention is administered in combination with a thiazolidinedione like troglitazone, ciglitazone, pioglitazone, rosiglitazone and the compounds disclosed in WO 97/41097 to Dr. Reddy's Research Foundation, especially 5-[[4-[(3,4-dihydro-3-methyl-4-oxo-2-quinazolinylmethoxy]phenyl]-methyl]-2,4-thiazolidinedione.

In a further preferred embodiment a compound of the invention is administered in combination with an a-glucosidase inhibitor like voglibose or acarbose.

In a further preferred embodiment a compound of the invention is administered in combination with a hepatic enzyme inhibitor of the kind described in WO 95/24391 which is hereby incorporated in its entirety by reference.

Furthermore, a compound of the invention may be administered in combination with an antihypertensive agent. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and a-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

Any novel feature or combination of features described herein is considered essential to this invention.

### EXAMPLES

The process for preparing compounds of formula (I) and preparations containing them is further illustrated in the following examples, which, however, are not to be construed as limiting. Where yields are reported, these are not optimised.

Hereinafter, TLC is thin layer chromatography, CDCl₃ is deuterio chloroform, CD₃OD is tetradeuterio methanol and DMSO-d₆ is hexadeuterio dimethylsulfoxide. The structures of the compounds were supported by either elemental analysis or NMR spectroscopy, where peaks assigned to characteristic protons in the title compounds are presented where appropriate. ¹H-NMR shifts (δ_{H}) are given in parts per million (ppm) down field from tetramethylsilane as internal reference standard. Mp: is melting point and is given in ° C. and is not corrected. HPLC-MS is high performance liquid chromatography-mass spectrometry, LCMS is liquid chromatography mass spectrometry, EIMS is electron impact mass spectrometry and ELS is evaporative light scattering.

Column chromatography was carried out using the technique described by W. C. Still et a/., J. Org. Chem. 43: 2923 (1978) on Merck silica gel 60 (Art. 9385). Rt is retention time. HPLC analyses were performed as described in the experimental section using either: HP1090 (analytical runs) or a Gilson HPLC system (preparative runs).

Unless otherwise indicated, the HPLC-MS analyses were performed on a PE Sciex API 100 LC/MS System using a WatersTM 3 mm×150 mm 3.5 μC-18 Symmetry column and positive ionspray with a flow rate at 20 μL/min. The column was eluted with a linear gradient of 5-90% A, 85-0% B and 10% C in 15 minutes at a flow rate of 1 ml/min (solvent A=acetonitrile, solvent B=water and solvent C=0.1% trifluoroacetic acid in water).

Compounds used as starting material are either known compounds or compounds, which can readily be prepared by methods known per se.

### Example 1

2-(2-(4,5-Dihydro-1H-imidazol-2-yl)-1-(3-methoxyphenyl)ethyl)pyridine

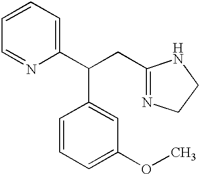

Magnesium (3.40 g, 0.14 mol) was suspended in tetrahydrofuran (40 ml) and a catalytic amount of iodine was added. To this suspension a solution of 3-bromoanisole (14.8 ml, 0.12 mol) in tetrahydrofuran (25 ml) was added dropwise. After the addition, the mixture was refluxed for 0.5 hour, cooled to room temperature and decanted into an addition funnel. A solution of 2-cyanopyridine (15 g, 0.144 mol) in tetrahydrofuran (60 ml) was cooled to −10° C. and the grignard solution was added dropwise at −10° C. and stirring was continued at room temperature for 16 hours. Hydrochloric acid (5N, 100 ml) was added and the mixture was stirred at room temperature for 3 hours. The mixture was made alkaline (pH=11) with portion wise addition of solid potassium hydroxide. The mixture was extracted with dichloromethane (3×200 ml), dried (MgSO₄) and concentrated in vacuo. The residue was purified by column chromatography on silica gel (800 ml) eluting with a mixture of ethyl acetate and heptane (1:1) which afforded 17.7 g (70%) of (3-methoxy-phenyl)pyidin-2-ylmethanone.

¹H-NMR (300 MHz, CDCl₃): δ 3.87 (3H, s), 7.15 (1H, ddd), 7.38 (1H, t), 7.49 (1H, ddd), 7.60-7.65 (2H, m), 7.90 (1H, td), 8.03 (1H, dt), 8.73 (1H, m).

TLC: R_{f}=0.53 (Silica gel; ethyl acetate/heptane 1:1)

Under nitrogen atmosphere sodium (1.15 g) was dissolved in ethanol (80 ml). Triethyl phosphonoacetate (11.2 g, 50 mmol) was added to the mixture followed by (3-methoxyphenyl)-pyidin-2-ylmethanone (7.41 g, 34.9 mmol) and the mixture was stirred at reflux for 2 hours. After cooling, the mixture was concentrated in vacuo. The residue was dissolved in dichloromethane (150 ml) and washed with aqueous potassium hydroxide (1N, 50 ml) and water (5×50 ml), dried (MgSO₄) and concentrated in vacuo to afford 10.2 g (100%) of 3-(3-methoxyphenyl-3-pyridine-2-yl)acrylic acid ethyl ester as a mixture of (E) and (Z) isomers.

¹H-NMR (300 MHz, CDCl₃, two isomers, selected data): δ 1.11 (3H, t), 3.75 (3H, s, major), 3.80 (3H, s, minor), 4.05 (2H, m), 6.45 (1H, s, major), 6.75-6.95 (3H, m), 7.03 (1H, d, minor), 7.19 (1H, s, minor), 7.20-7.35 (3H, m), 7.54 (1H, dt, minor), 7.71 (1H, dt, major), 8.63 (1H, m, major+minor).

TLC: R_{f}=0.58 & 0.42 (silica gel; ethyl acetate/heptane 1:1)

3-(3-Methoxyphenyl-3-pyridine-2-yl)acrylic acid ethyl ester (10.2 g, 35.9 mmol) was dissolved in ethanol (100 ml) and under a nitrogen atmosphere 10% palladium on carbon (1 g) was added. The mixture was hydrogenated at room temperature and atmospheric pressure. The mixture was filtered and concentrated in vacuo. The residue was purified by column chromatography on silica gel (800 ml) eluting with a mixture of ethyl acetate and heptane (1:1) to afford 7.55 g (74%) of 3-(3-methoxyphenyl-3-pyridine-2-yl)propionic acid ethyl ester as an oil.

¹H-NMR (300 MHz, CDCl₃): δ 1.15 (3H, t), 2.95 (1H, dd), 3.46 (1H, dd), 3.78 (3H, s), 4.07 (2H, q), 4.63 (1H, dd), 6.73 (1H, dd), 6.9 (3H, m), 7.10 (1H, m), 7.20 (2H, m), 7.56 (1H, dt), 8.55 (1H, m).

TLC: R_{f}=0.57 (Silica gel; ethyl acetate/heptane 1:1)

A solution of trimethylaluminium in toluene (2M, 25 ml, 50 mmol) under an argon atmosphere was cooled to −10° C. and ethylenediamine (3.3 ml, 50 mmol) was added dropwise maintaining the temperature below −5° C. The resulting mixture was stirred at room temperature for 2 hours. 3-(3-Methoxyphenyl-3-pyridine-2-yl)propionic acid ethyl ester (6.50 g, 22.8 mmol) was added and the resulting mixture was stirred at reflux for 18 hours. After cooling, the mixture was poured into a mixture of methanol (100 ml), dichloromethane (100 ml), and water (25 ml), stirred at room temperature for 1 hour, filtered through Celite® and concentrated in vacuo. The residue was dissolved in ethyl acetate and refluxed for 0.5 hour, cooled to room temperature, filtered and concentrated in vacuo. The residue was purified by column chromatography on silica gel (800 ml) eluting with methanol containing 2.5% triethylamine followed by bulb-to-bulb distillation (250° C.,3×10⁻² mbar) to afford 2.32 g (36%) of the title compound as an oil.

¹H-NMR (300 MHz, CDCl₃): δ 2.90 (1H, dd), 3.30 (1H, dd), 3.75 (3H, s), 4.50 (1H, dd), 6.72 (1H, dd), 6.90 (2H, m), 7.1-7.2 (3H, m), 7.55 (1H, dt), 8.55 (1H, m).

Calculated for C₁₇H₁₉N₃O: C, 72.57%; H, 6.81%; N, 14.93%.

Found: C, 72.32%; H, 7.08%; N, 14.66%;

### Example 2

(−)-2-(2-(4,5-Dihydro-1H-imidazol-2-yl)-1-(3-methoxyphenyl)ethyl)pyridine

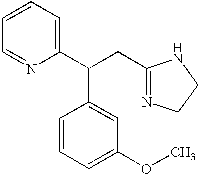

The product of Example 1 was separated into pure enantiomers using a Chiralcel® OD column (250-20 mm, Daicel) eluted with a mixture of heptane/2-propanol/diethylamine (80:20:0.1) at a flow rate of 6 ml/min. The compound was dissolved in a mixture of 2-propanol/diethylamine/heptane (44:0.2:56), 50 mg/ml, injected in portions of 20 mg and detected at 225 and 265 nm. The two enantiomers (A and B) eluted at T_{R} 28-40 min (A) and T_{R} 43-55 min (B), respectively were collected (10 ml/fraction) and pooled. The purity of the enantiomers was determined using a Chiraicel® OD (250-4.6 mm, Daicel) column eluted with a mixture of heptane/2-propanol/diethylamine (80:20:0.1) at a flow rate of 0.8 ml/min, T_{R}(A): 11.5 min and T_{R}(B): 16.3 min, detected at 225 and 265 nm. The title compound (compound (Ex 2) was obtained as the first eluting enantiomer (enantiomer A): [α]²⁰_{D}=−91.5° (c=0.29, MeOH).

### Example 3

(+)-2-(2-(4,5-Dihydro-1H-imidazol-2-yl)-1-(3-methoxyphenyl)ethyl)pyridine

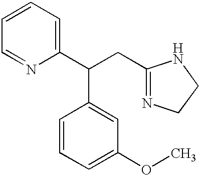

The title compound was obtained as the second eluting enantiomer (enantiomer B) as described in Example 2.

[α]²⁰_{D}=+91.5° (c=0.22, MeOH)

### Example 4

Preparation of Intermediates

a) Pyridine-3-yl-thiophene-2-yl-methanone.

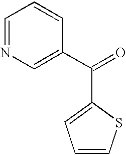

Butyl lithium (10.0 ml, 1.6 M in hexane, 16 mmol) was added to dry ether (30 ml) under nitrogen atmosphere at a temperature of −78° C. 2-bromothiophene (2.44 g, 15 mmol) in dry ether (25 ml) was added dropwise at a temperature below −72° C. The mixture was subsequently heated to −30° C. for 20 min. Further cooling to −78° C. followed by addition of 3-cyanopyridine (1.56 g, 15 mmol) in dry ether (30 ml) at a temperature <−72° C. over 10 min. resulted in a red solution with some precipitate.

The mixture was further cooled to −78° C. for 20 min. followed by heating to 0° C. for 10 min. HCl (6 N) was added to pH 1, followed by stirring for 30 min. KOH was then added to pH 10. Ethyl acetate (50 ml) and water (10 ml) was added and the organic phase was separated, dried with MgSO₄ and evaporated to dryness resulting in yellow oil.

The crude product was treated with HCl (6 N, 50 ml) for 1 hour and the pH was then adjusted to 10 by addition of solid KOH. Extraction, drying, and evaporation as described above resulted in yellow crystals (2.4 g) of pyridine-3-yl-thiophene-2-yl-methanone, Mp: 90-91.5° C. MS: M+=189.

The following intermediates were prepared from the appropriate starting materials using the same procedure as described under a):

b) Pyridine-3-yl-thiophene-3-yl-methanone.

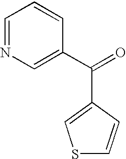

From 3-bromothiophene (2.44 g, 15 mmol) and 3-cyanopyridine (1.56 g, 15 mmol). Yield of pyridine-3-yl-thiophene-3-yl-methanone: 2.53 g, mp: 71.5-72° C., MS: M+=189.

c) Pyridine-4-yl-thiophene-2-yl-methanone.

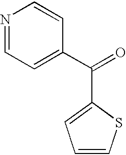

From 2-bromothiophene (2.44 g, 15 mmol) and 4-cyanopyridine (1.56 g, 15 mmol). Yield of pyridine-4-yl-thiophene-2-yl-methanone: 2.51 g, mp: 101° C., MS: M+=189.

d) Pyridine-4-yl-thiophene-3-yl-methanone.

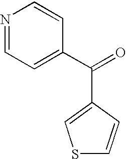

From 3-bromothiophene (2.44 9, 15 mmol) and 4-cyanopyridine (1.56 9, 15 mmol). Yield of pyridine-4-yl-thiophene-3-yl-methanone: 1.91 9, mp: 71.5-72° C., MS: M+=189.

e) Pyridine-2-yl-thiophene-3-yl-methanone.

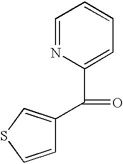

From 3-bromothiophene (20 g, 120 mmol) and 2-cyanopyridine (12.5 g, 120 mmol). Yield of pyridine-2-ylthiophene-3-ylmethanone: 4.9 g, (oil). MS: M+1=190.

### Example 5

3-[2-(4,5-Dihydro-1H-imidazol-2-yl)-1-thiophene-2-yl-ethyl]-pyridine

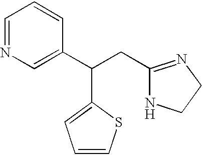

Na (0.12 g, 5 mmol) was dissolved in dry ethanol (10 ml), the solution was cooled to −5° C. and triethyl phosphonoacetate (1.13 g, 5 mmol) in ethanol (1 ml) was added and the mixture stirred for 30 min. Pyridine-3-yl-thiophene-2-yl-methanone (0.76 g, 4 mmol) in ethanol (1 ml) was added and the mixture was heated to 55° C. for 3 days. The reaction mixture was then evaporated to dryness to give a crude Z-E mixture of 3-pyridine-3-yl-3-thiophene-2-yl-acrylic acid ethyl esters as an oil (1.2 g).

The crude product was dissolved in dry ethanol (40 ml) and hydrogenated over Pd/C (10%, 0.25 g) at 20 psi overnight. The reaction mixture was filtered through filter aid and evaporated to dryness.

The crude product was purified on silica gel using dichloromethane/MeOH (19:1) as the eluent. Yield of hydrogenated product: 265 mg. The product contained a small amount of starting material. LC/MS: M+1=262. This product was used without further purification in the following reaction.

Trimethyl aluminium (2 N in toluene, 0.75 ml, 1.5 mmol) was cooled to −10° C., ethylenediamine (0.1 ml, 1.5 mmol) was added slowly and the mixture was stirred for 30 min. The crude product from above (260 mg) in dry toluene (10 ml) was added dropwise over 30 min resulting in a yellow solution.

The mixture was heated at reflux temperature for 1 h resulting in a red solution. Subsequently the solution was cooled to 0° C. and water (1 ml), MeOH (2 ml) and dichloromethane (2 ml) was added and the mixture heated to 40° C. for 20 min resulting in a blue solution. The organic phase was separated and the water phase was extracted 3 times with dichloromethane (3 ml). The combined organic phases were dried (Na₂SO₄) and evaporated to dryness.

Purification on silica gel using dichloromethane/MeOH/triethylamine (45:5:3) resulted in 3-[2-(4,5-dihydro-1H-imidazol-2-yl)-1-thiophene-2-yl-ethyl]-pyridine as an oil (70 mg), which was isolated by evaporation of the solvent from the fourth eluting fraction.

LC/MS: M+1=258, ELS purity: 99%.

### Example 6

4-[2-(4,5-Dihydro-1H-imidazol-2-yl)-1-thiophene-3-yl-ethyl]-pyridine

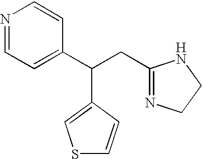

The title compound was prepared from pyridine-4-yl-thiophene-3-yl-methanone using corresponding amounts of the remaining reagents as described in Example 5. Yield of 4-[2-(4,5-dihydro-1H-imidazol-2-yl)-1-thiophene-3-yl-ethyl]-pyridine purified on a silica gel column: 80 mg of an oil isolated from the fourth eluting fraction. MS: M+=257.

### Example 7

4-[2-(4,5-Dihydro-1H-imidazol-2-yl)-1-thiophene-2-yl-ethyl]-pyridine

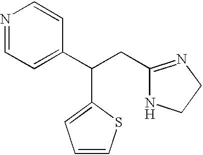

The title compound was prepared from pyridine-4-yl-thiophene-2-yl-methanone using corresponding amounts of the remaining reagents as described in Example 5. The resulting product was purified twice on a silica gel column. In the second purification EtOAc/MeOH/NH₃ (25%) (4:1:1) was used as the eluent. 4-[2-(4,5-dihydro-1H-imidazol-2-yl)-1-thiophene-2-yl-ethyl]-pyridine (1 3 mg) was isolated as an oil. MS: M+=257.

### Example 8

2-[2-(4,5-Dihydro-1H-imidazol-2-yl)-1-thiophene-3-yl-ethyl]-pyridine

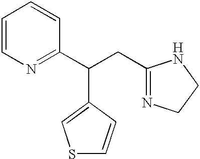

Na (0.8 g, 35 mmol) was dissolved in dry ethanol (20 ml), the solution was cooled to −5 oC and triethyl phosphonoacetate (7.8 g, 5 mmol) was added followed by pyridine-2-ylthiophene-3-ylmethanone (4.9 g, 26 mmol) and the mixture was stirred at room temperature for 2 hours. The reaction mixture was then evaporated to dryness and partitioned between water (100 ml) and dichloromethane (2×150 ml). The combined organic extracts were dried (Na2SO4) and concentrated in vacuo to afford a Z-E mixture of 3-pyridine-2-yl-3-thiophene-3-ylacrylic acid ethyl ester as an oil (9.14 g) containing residual triethyl phosphonoacetate (approx. 2.7 g as determined by NMR spectroscopy). The crude product was dissolved in dry ethanol (100 ml) and hydrogenated over Pd/C (10%, 50% water, 1.0 g) at atmospheric pressure for 6 hours. More Pd/C (1 g) was added and hydrogenated at atmospheric pressure for 6 hours. The mixture was filtered and Pd/C (1 g) was added and hydrogenated at atmospheric pressure for 6 hours and at 30 psi overnight and at 30-35 bar overnight. The reaction mixture was filtered through filter aid and evaporated to dryness. The crude product was purified on silica gel using ethyl acetate:heptane (2:1) as the eluent. This afforded 1.0 g 3-pyridine-2-yl-3-thiophene-3-ylpropionic acid ethyl ester. This product was used without further purification in the following reaction.

3-Pyridine-2-yl-3-thiophene-3-ylpropionic acid ethyl ester (1.0 g, 3.8 mmol) was treated with triethylaluminium as described above Ex 5 resulting in a crude oil (1 g). 100 mg of this oil was purified by preparative TLC on silica gel using EtOAc/MeOH/NH₃ (25%) (4:1:1) as the eluent. 2-[2-(4,5-Dihydro-1H-imidazol-2-yl)-1-thiophene-3-ylethyl]pyridine was isolated from the second eluting fraction (32 mg). LC/MS: M+1=258, ELS purity 99%.

### Example 9

Separation of the Enantiomers of 4-[2-(4,5-Dihydro-1H-imidazol-2-yl)-1-thiophene-3-yl-ethyl]-pyridine

Racemic 4-[2-(4,5-Dihydro-1H-imidazol-2-yl)-1-thiophene-3-yl-ethyl]-pyridine was separated into its enantiomers on a chiral column (Chiralcel® OD), eluent: heptane/2-propanol/diethylamine (50:50:0,1).

The first eluting enantiomer of 4-[2-(4,5-Dihydro-1H-imidazol-2-yl)-1-thiophene-3-yl-ethyl]-pyridine had retention time: 13,9 min, purity: >99,3% ee.

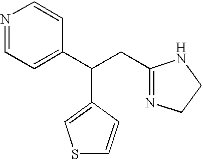

The second eluting enantiomer of 4-[2-(4,5-Dihydro-1H-imidazol-2-yl)-1-thiophene-3-yl-ethyl]-pyridine had retention time: 17,6 min, purity: >93,0% ee

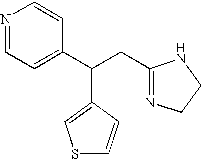

### Example 10

Separation of the Enantiomers of 2-[2-(4,5-Dihydro-1H-imidazol-2-yl)-1-thiophene-3-yl-ethyl]-pyridine

Racemic 2-[2-(4,5-Dihydro-1H-imidazol-2-yl)-1-thiophene-3-yl-ethyl]-pyridine was separated into its enantiomers on a chiral column (Chiralpak® AD, Chiralcel® OD), eluent: heptane/2-propanol/diethylamine (50:50:0,1).

The first eluting enantiom er of 2-[2-(4,5-Dihydro-1H-imidazol-2-yl)-1-thiophene-3-yl-ethyl]-pyridine had retention time: 10.2 min; purity: 80.1% ee.

The second eluting enantiomer of 2-[2-(4,5-Dihydro-1H-imidazol-2-yl)-1-thiophene-3-yl-ethyl]-pyridine had retention time: 14.4 min.; purity: 99.5% ee.

### Example 11

2-[2-(4,5-Dihydro-1H-imidazol-2-yl)-1-(5-iodo-2,3-dihydro-benzofuran-7-yl)-ethyl]-pyridine

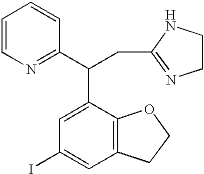

2-[2-(4,5-Dihydro-1H-imidazol-2-yl)-1-(2,3-dihydro-benzofuran-7-yl)-ethyl]-pyridine (600 mg, 2 mmol) was dissolved in glacial acetic acid (10 mL), iodine monochloride (800 mg, 4.9 mmol) dissolved in glacial acetic acid (10 mL) was added over 3 min. under nitrogen. The mixture was stirred at room temperature overnight, a yellow precipitate was filtered off, dissolved in dichloromethane which subsequently was extracted with NaOH (1M). The dichloromethane phase was dried (MgSO₄) and evaporated to dryness to give an oil which crystallised on addition of acetone.

Yield: 400 mg (47%), LCMS: m/z: 420 (M+1)⁺, retention time: 3.65 min, ELS purity: 87%.

This compound was subsequently separated in enantiomers on Chiralpak® AD, using 2-propanol:heptane:diethylamine, 10:90:0.1 as eluent,

(+)-2-[2-(4,5-Dihydro-1H-imidazol-2-yl)-1-(5-iodo-2,3-dihydro-benzofuran-7-yl)-ethyl]-pyridine

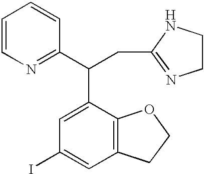

Yield: 47%, retention time: 16.3 min. Purity:>99,7% ee. Optical rotation: +24.2°, (ethanol: acetonitrile 75:25, 25° C.).

(−)-2-[2-(4,5-Dihydro-1H-imidazol-2-yl)-1-(5-iodo-2,3-dihydro-benzofuran-7-yl)-ethyl]-pyridine

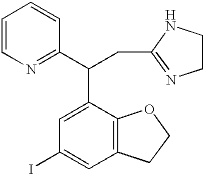

Yield: 45%, retention time: 23.0 min. Purity: 99,0% ee. Optical rotation: -27,60, (ethanol:acetonitrile 75:25, 25° C.).

### Example 12

(Diethoxy-phosphorylmethyl)-4,5-dihydro-imidazole-1-carboxylic Acid Tert-Butyl Ester

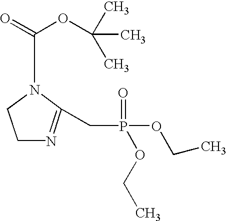

Diethyl cyanomethylphosphonate (50 g) was converted to the imidate hydrochloride by addition of ethanol (22 mL) and ether (300 mL), cooling to 0° C. followed by saturation with HCl (g).

The mixture was kept in a closed bottle for 48 h, and subsequently evaporated to dryness. The resulting imidate hydrochloride was added portionwise to a mixture of ethylene-1,2-diamine (16.6 g) in ethanol (140 mL) and heated to 40° C. for 1 h. Ethanol (350 mL) was added and the mixture concentrated yielding 90% of 2-imidazolinylmethyl diethyl phosphonate hydrochloride. This crude product was dissolved in dichloromethane (150 mL) and triethylamine (140 mL) and reacted with BOC-anhydride (65 g) added in 3 portions over 30 min. The mixture was stirred at room temperature for 20 h. Water was added and the organic phase was isolated and evaporated to dryness.

The crude product was purified on a SiO₂ column using 95:5 dichloromethane:methanol as eluent.

Yield: 23.5 g ¹H NMR (CDCl₃) ppm:1.30 (6H,t); 1.48 (9H,s); 3.50 (2H,d); 3.75 (4H,m);4.14 (4H,d q).

### Example 13

2-(Diethoxy-phosphorylmethyl)-4,5-dihydroimidazole-1-carboxylic Acid Benzyl Ester

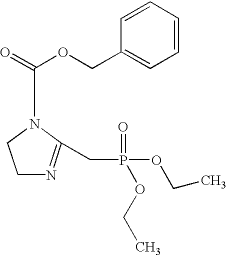

2-imidazolinylmethyl diethyl phosphonate hydrochloride (40 g) was dissolved in dichloromethane (500m L), the mixture was cooled to 0° C., triethylamine (140 mL) was added followed by benzyloxycarbonyl chloride (41 mL in dichloromethane (60 mL). The resulting mixture was stirred at room temperature for 16 h and then slowly poured on ice (200 g), extracted with dichloromethane and concentred in vacuo. The resulting mixture was attempted purified in silica gel using ethyl acetate and ethanol as eluent, this procedure however resulted in partly hydrolysis of the product. Purification on Al₂O₃ (neutral) with ethyl acetate as eluent resulted in 2-(diethoxy-phosphory lmethyl)-4,5-dihydro-imidazole-1-carboxylic acid benzyl ester (10 g). ¹H NMR (CDCl₃) ppm:1.30 (6H, t); 3.55 (2H,d); 3.80 (4H,m); 4.14 (4H,d q); 5.18 (2H,s); 7.35 (5H, m).

### Example 14

Separation of the Enantiomers of 4-[2-(4,5-Dihydro-1H-imidazol-2-yl)-1-thiophen-2-yl-ethyl]-pyridine

Racemic 4-[2-(4,5-Dihydro-1H-imidazol-2-yl)-1-thiophen-2-yl-ethyl]-pyridine was separated on Chiralcel® OD using 2-propanol:heptan:diethylamine (50:50:0.1) as eluent.

(+)-4-[2-(4,5-Dihydro-1H-imidazol-2-yl)-1-thiophen-2-yl-ethyl]-pyridine

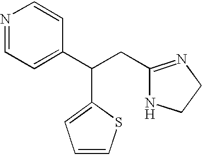

Yield: 20%, retention time: 10.4 min. Purity: >98,9% ee. Optical rotation: +28,7 (2-propanol:heptan:diethylamine (50:50:0.1)).

(−)-4-[2-(4,5-Dihydro-1H-imidazol-2-yl)-1-thiophen-2-yl-ethyl]-pyridine

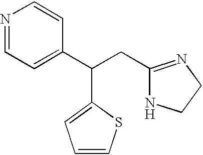

Yield: 24%, retention time: 1.0 min, purity: >98,2% ee. Optical rotation: +26,3° (2-propanol:heptan:diethylamine (50:50:0.1)).

### Example 15

Z/E-3-[2-(4,5-Dihydro-1H-imidazol-2-yl)-1-thiophen-3-yl-vinyl]-pyridine

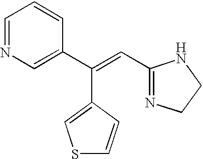

Preparation from 3-thienyl-3-pyridyl ketone (5 g) and 2-(diethoxy-phosphorylmethyl)-4,5-dihydro-imidazole-1-carboxylic acid tert-butyl ester (8.45 g) by means of BuLi (20 mL, 1.6 N in hexane) in THF using the method described in Example 18 gave a yield of the BOC-protected compound in a yield of 98% (9.2 g).

2 g of this was de-protected in HCl (4 mL, 3N) and ethyl acetate (4 mL) by stirring overnight; resulting in the target compound (1.2 g, 90%). LCMS m/z: 256(M+1)⁺; retention time: 0.59 min and 0.85 min, respectively (ratio 1/3).

### Example 16

3-[2-(4,5-Dihydro-1H-imidazol-2-yl)-1-thiophen-3-yl-ethyl]-pyridine

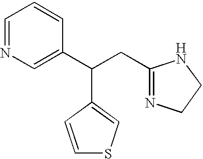

Z/E-3-[2-(4,5-Dihydro-1H-imidazol-2-yl)-1-thiophen-3-yl-vinyl]-pyridine (107) 8 (0.52 g) was hydrogenated over Pd/C (10%, 50 mg) in ethanol (10 mL) using a pressure of 30 psi for 4 h. The reaction mixture was filtered through filter aid and evaporated to dryness to give 360 mg (65%) of the target compound. LCMS: m/z: 258 (M+1)⁺, retention time: 0.35 min, ELS purity: 97%. ¹³C NMR (CDCl₃) ppm:167.4, 149.5, 148.4, 143.0, 138.8, 135.7, 127.6, 126.8, 124.0, 121.7, 57.7, 48.1, 41.8, 35.1.

### Example 17

1-{2-[2-(3-Methoxy-phenyl)-2-pyridin-2-yl-ethyl]-4,5-dihydro-imidazol-1-yl}-ethanone

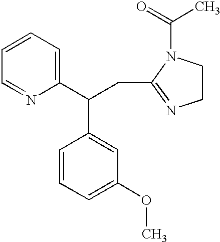

2-(2-(4,5-Dihydro-1H-imidazol-2-yl)-1-(3-methoxyphenyl)ethyl)pyridine (exp1) (250 mg, 0.89 mmol), was dissolved in THF (10 mL), triethylamine (0.89 mmol) in dichloromethane (2 mL) was added, the mixture was cooled on ice and acetyl chloride (0.89 mmol) in dichloromethane (2 mL) was added.

After 30 min a gas evolution was observed. The mixture was heated to reflux for 4 h, water (5 mL) was added and the mixture extracted with dichloromethane (3×2 mL). The organic layers were collected, dried over MgSO₄ and evaporated to dryness yielding a red-yellow oil.

The crude product was purified on silicagel column using dichloromethane/MeOH (9/1) as eluent, yielding an oil (85 mg, 29%) LCMS: m/z 324 (M+1)⁺, retention time: 3.08 min; ELS purity: 85%. EIMS: m/z 323 (M+). ¹³C NMR (CDCl₃), ppm: 167.8; 162.5; 160.2; 159.5; 149.0; 145.0; 136.2; 129.3; 123.3; 121.3; 120.6; 114.0; 111.9; 55.1; 52.7; 50.0; 47.5; 36.6; 25.3.

### Example 18

Z,E-3-(3-Methoxy-phenyl)-3-pyridin-2-yl-acrylonitrile

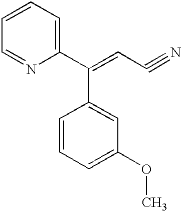

A NaH suspension in mineral oil (60%) (0.036 mol, 1.44 g), previously washed with heptane under N₂ flow was dissolved in dry THF (30 mL).The mixture was cooled to 0° C. Diethyl cyanomethylphosphonate (0.036 mol, 6.5 g) dissolved in THF (10 mL) was added dropwise. Stirring at 0° C. for 45 min. (3-methoxyphenyl) pyridin-2-yl-methanone (0.024 mol, 5.11) in THF (40 mL) was added slowly. The reaction mixture was heated to 80° C. and refluxed during 2 h. TLC showed starting material left. In order to finish the reaction, more diethyl cyanomethylphosphonate (0.017 mol, 3.04 g) and NaH suspension (60%) (0.028 mol, 0.672 g) mixture was added. The resulting mixture was refluxed again during 1 h. It was cooled to room temperature and the solvent was evaporated. The residue was dissolved in dichloromethane and extracted with H₂O (150 mL). The organic phase was dried over MgSO₄, filtered and evaporated to dryness. The crude oil was purified by Flash 40 silica column chromatography using EtOAc/n-heptane (2:1) as eluent, to give a green solid. The Z/E mixture was isolated (5.43 g, 96%). Diastereomeric ratio: 60/30. Peaks corresponding to the 2 isomers were observed in the ¹H-NMR. ¹H-NMR (001006, 200 MHz) (CDCl₃, ppm): 3.78 (s, 3H)*; 3.81 (s, 3H)**; 5.89 (s, 1H)*; 6.69 (s, 1H)**; 6.81-7.50 (m, 6H); 7.64 (ddd, 1H)**; 7.80 (ddd, 1H)*; 8.66 (m, 1H)**; 8.75 (m, 1H)* (peaks marked ** belonging to the major isomer and peaks marked * belonging to the minor isomer).

### Example 19

3-(3-Methoxy-phenyl)-3-pyridin-2-yl-propionitrile

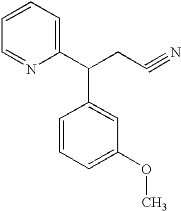

A mixture of Z/E-3-(3-methoxy-phenyl)-3-pyridin-2-yl-acrylonitrile (0.021 mol, 5 g) and Pd/C (10%, 0.4 g) in EtOH (100 mL) was hydrogenated at 60 psi, at room temperature. After 60 h the resulting reaction mixture was filtered through Celite®, dried over MgSO₄ and evaporated to give a brown oil, (4.1 g, 80%). which was purified on silica gel column using EtOAc/n-heptan (1:1) as eluent.

¹H-NMR (200 MHz) (CDCl₃, ppm): 3.06 (dd, 1H); 3.37 (dd, 1H); 3.75 (s, 3H); 4.40 (t, 1H); 6.75-6.90 (m, 3H); 7.09-7.30 (m, 3H); 7.58 (m, 1H); 8.59 (m, 1H).

### Example 20

2-[2-(4,4-Dimethyl-4,5-dihydro-1H-imidazol-2-yl)-1-(3-methoxy-phenyl)-ethyl]-pyridine

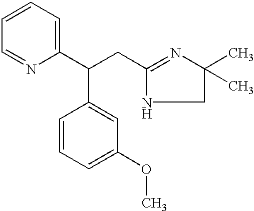

2-Methyl-propane-1,2-diamine monotosylate (1.5 mmol, 390 mg) and 3-(3-methoxy-phenyl)-3-pyridin-2-yl-propionitrile (1 mmol, 240 mg) were reacted by heating to 160° C. for 7 h. The reaction mixture was extracted between EtOAc (40 mL) and NaOH (1M, 40 mL). Further purification of the organic fraction by re-extraction with NaOH 1 M. The organic layer was rification of the organic fraction by re-extraction with NaOH 1M. The organic layer was dried over MgSO₄, filtered and evaporated to give the target product (100 mg, 32%). LC-MS (m/z: 310 (M+1)⁺) ¹H-NMR (CDCl₃, ppm): 1.00 (s, 3H); 1.05 (s, 3H); 2.89 (dd, 1H); 3.15 (s, 2H); 3.22 (dd, 1H); 4.50 (dd, 1H); 6.70-7.20 (m, 6H); 7.56 (m, 1H); 8.55 (m, 1H). ¹³C-NMR (CDCl₃, ppm): 27.49, 27.59, 29.06, 33.91, 50.38, 54.54, 62.00, 111.48, 113.10, 119.62, 121.07, 123.26, 128.91, 135.96, 143.90, 148.20.

### Example 21

2-[1-(3-Methoxy-phenyl)-2-(1-methyl-4,5-dihydro-1H-imidazol-2-yl)-ethyl]-pyridine

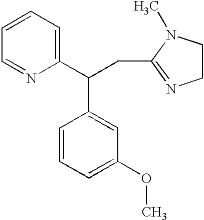

2-(2-(4,5-Dihydro-1H-imidazol-2-yl)-1-(3-methoxyphenyl)ethyl)pyridine (0.89 mmol, 250 mg) dissolved in dry THF (2 mL) was added to a suspension of NaH (0.89 mmol, 22 mg) in THF (2 mL) the mixture was stirred at room temperature for 30 min followed by heating to reflux for 5 min. Subsequently the mixture was cooled on acetone /dry ice and methyl iodide ((0.89 mmol) in THF (2 mL) was added.

The mixture was heated to room temperature and stirred overnight, NaOH (3 mL, 4 N) was added and the mixture extracted with dichloromethane. The combined organic phases were dried (MgSO₄) and evaporated to dryness. 200 mg of crude product was isolated. A small fraction of this was purified on Xterra MS C_{18, 5} μM 19×100 mm column using CH₃CN/H2O/0.01% trifluoroacetic acid, grad 10-100% CH₃CN/11 min for the elution.

LCMS: retention time: 1.81 min, ELS purity:100%, m/z 296 (M+1)⁺.

### Example 22

2-[1-(3-Methoxy-phenyl)-2-(1-propyl-4,5-dihydro-1H-imidazol-2-yl)-ethyl]-pyridine

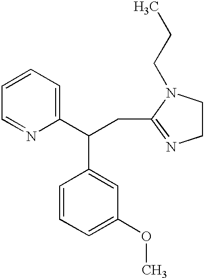

2-(2-(4,5-Dihydro-1H-imidazol-2-yl)-1-(3-methoxyphenyl)ethyl)pyridine (0.89 mmol, 250 mg) dissolved in dry THF (2 mL) and NaH (0.89 mmol, 22 mg) in THF (2 mL) were reacted with 1-propyl iodide (0.89 mmol) as described above. A crude product (100 mg) of yellow oil was isolated. This was purified as described above to give the target compound. LCMS: retention time: 2.32 min, ELS purity: 100%, m/z 324 (M+1)⁺.

### Example 23

2-[1-(3-Methoxy-phenyl)-2-(4-methyl-4,5-dihydro-1H-imidazol-2-yl)-ethyl]-pyridine

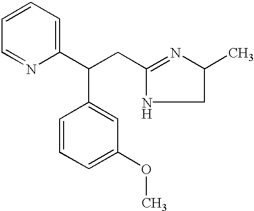

This compound was prepared from 3-(3-Methoxy-phenyl)-3-pyridin-2-yl-propionitrile (1.2 mmol, 280 mg) and the monotosylate of propane-1,2-diamine (2.3 mmol, 579 mg) as described in Example 20.

Further purification of the organic fraction by re-extraction with NaOH 1M. The organic layer was dried (150 mg, 42%). Peaks corresponding to the two tautomers were observed in the ¹H-NMR and ¹³C-NMR. LC-MS: retention time: 1.76 min; m/z: 296 (M+1)⁺; ¹H-NMR (CDCl₃, ppm): 0.81 (d), 0.87 (d), (3H); 2.95-2.75 (m, 2H); 3.19 (dd, 1H); 3.46 (t, 1H); 3.65 (s, 3H); 3.69 (m, 1H); 4.45 (dd, 1H); 6.60-6.66 (m, 1H); 6.80-6.84 (m, 2H); 6.96-7.13 (m, 3H); 7.44 (m, 1H); 8.45 (m, 1H). ¹³C-NMR (CDCl₃, ppm): 21.74, 21.94, 34.91, 51.29, 51.42, 55.45, 56.65, 57.51, 112.37, 114.07, 120.49, 122.01, 124.02, 129.88, 136.86, 145.07, 145.16, 149.16, 160.00, 162.36, 165.89.

### Example 24

2-[2-(1-Ethyl-4,5-dihydro-1H-imidazol-2-yl)-1-(3-methoxy-phenyl)-ethyl]-pyridine

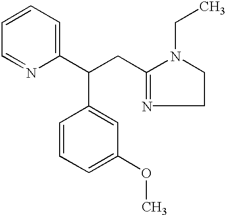

This compound was prepared from 3-(3-methoxy-phenyl)-3-pyridin-2-yl-propionitrile (0.94 mmol, 224 mg) and the monotosylate of N-ethylethane-1,2-diam ine (1.8 mmol, 468 mg) as described for compound example 20. The crude product obtained as an oil, (176 mg, 61%), was purified by semipreparative HPLC (eluent: acetonitrile) to give a yellow oil. LC-MS: retention time: 2.06 min, m/z: 310 (M+1)⁺; ¹H-NMR (CDCl₃, ppm): 1.07 (t, 3H); 2.90 (dd, 1H); 3.14-3.44 (m, 5H); 3.63 (m, 1H); 4.85 (dd, 1H); 6.72 (m, 1H); 6.93-6.97 (m, 2H); 7.06-7.23 (m, 3H); 7.54 (ddd, 1H); 8.53 (m, 1H). ¹³C-NMR (CDCl₃, ppm): 13.52; 29.71; 32.16; 41.10; 49.27; 50.03; 55.22; 112.11; 113.83; 120.39; 121,56; 124.12; 129.46; 136.46; 144.94; 148.78; 159.63; 161.75; 166.71.

### Example 25

2-[2-(3-Methoxy-phenyl)-2-pyridin-2-yl-ethyl]-3a,4,5,6,7,7a-hexahydro-1H-benzimidazole

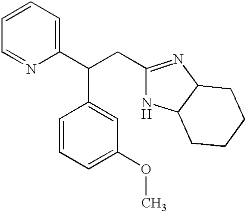

trans-Cyclohexane-1,2-diamine monotosylate (2.2 mmol, 630 mg) and 3-(3-methoxy-phenyl)-3-pyridin-2-yl-propionitrile (1.08 mmol, 258 mg) were reacted by heating to 160° C. for 24 h. The reaction mixture was extracted between EtOAc (40 mL) and NaOH (1M, 40mL). Further purification of the organic fraction by re-extraction with NaOH 1M. The organic layer was dried over MgSO₄, filtered, and evaporated to give the crude product (290 mg, 80%). Purification by semi-preparative HPLC gave a more pure batch of (53.5 mg). Peaks corresponding to the two tautomers were observed in the ¹H-NMR and ¹³C-NMR spectra. LC-MS: retention time: 2.21 min; m/z: 336 (M+1)⁺; ¹H-NMR (CDCl₃, ppm): 0.83-2.10 (m, broad, 8H); 2.71 (m, broad, 1H); 2.94 (m, 1H); 3,32 (m, 1H); 3.53 (m, broad,1H); 3.75 (s, 3H); 4.54 (m, 1H); 4.79 (s, broad, 1H,NH); 6.72 (m, 1H); 6.86-6.94 (m, 2H); 7.07-7.23 (m, 3H); 7.55 (m, 1H); 8.55 (m, 1H). ¹³C-NMR (CDCl₃, ppm): 20.71(CH₂); 21.04(CH₂); 24.91(CH₂); 27.96(CH₂); 29.69(CH₂); 30.67(CH₂); 34.77(CH₂); 35.30(CH₂); 35.75(CH₂); 50.90(CH); 51.11 (CH); 55.15(CH3); 59.30(CH); 60.14(CH); 69.19(CH); 112.16(CH); 113.69(CH); 120.23(CH); 121.70(CH); 123.94(CH); 129.55(CH); 136.58(CH); 144.56; 148.85(CH); 159.69; 161.98; 166.67.

### Example 26

Z,E-4-[2-(4,5-Dihydro-1H-imidazol-2-yl)-1-(3-methoxy-phenyl)-vinyl]-pyridine

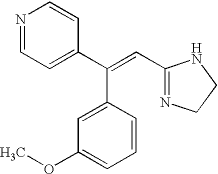

Z,E-2-[2-(3-methoxy-phenyl)-2-pyridin-4-yl-vinyl]-4,5-dihydro-imidazole-1-carboxylic acid tertbutyl ester (431 mg,1.13 mmol) was de-BOC'ed in HCl (5 mL, 3 N) and ethyl acetate (5 mL) by stirring 2 days at room temperature. pH was adjusted to 10 (NaOH,10 M) the organic phase was separated, dried (MgSO₄) and evaporated to dryness to give a yellow oil (120 mg, 38%). LCMS: m/z 280 (M+1)⁺; retention time: 1.21 min. ¹H NMR (CDCl₃) ppm: 3.53(s,4H); 3.76/3.82(singlets ratio 7/3, 3H); 4.89(s,1H); 6.77-7.03(m,4H); 7.15-7.43(m,3H); 8.52-8.69(m,2H).

### Example 27

2-[2-(3-Methoxy-phenyl)-2-pyridin-2-yl-ethyl]-1,4,5,6-tetrahydro-pyrimidine

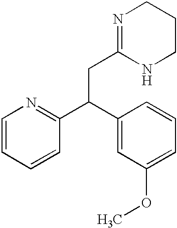

Propane-1,3-diamine monotosylate, (492 mg, 2 mmol) and 3-(3-methoxy-phenyl)-3-pyridin-2-yl-propionitrile, (1.17 mmol, 280 mg) were reacted by heating to 160° C. for 8 h. The reaction mixture was extracted between EtOAc (40 mL) and NaOH (1M, 40 mL). Further purification of the organic fraction was performed by re-extraction with NaOH 1M. The organic layer was dried over MgSO₄, filtered and evaporated to give the crude product (190 mg, 55%), which was purified by preparative HPLC.LCMS: retention time: 1.71 min; m/z: 296 (M+1)⁺.

### Example 28

2-[2-(4,5-Dihydro-thiazol-2-yl)-1-(3-methoxy-phenyl)-ethyl]-pyridine

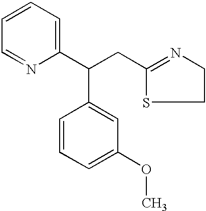

A mixture of 3-(3-methoxy-phenyl)-3-pyridin-2-yl-propionitrile (1.09 mmol, 260 mg), 2-aminoethanthiol hydrochloride (5.45 mmol, 610 mg), and zinc chloride (100 mg) was heated at reflux (132° C.) in chlorobenzene (15 mL) for 18 h. TLC and an NMR spectrum showed starting material left. In order to finish the reaction zinc chloride (90 mg) and 2-aminoethanthiol hydrochloride (300 mg) in chlorobenzene (15 mL) were added. The resulting mixture was refluxed overnight. The reaction mixture was concentrated and diluted with EtOAc. The solution was extracted twice with saturated NaHCO₃. The organic fraction was dried over MgSO₄, filtered and the solvent evaporated. The crude product was purified by column-chromatography on silica gel using EtOAc as eluent to give 70 mg (21%). LCMS: retention time: 2.30 min; m/z: 299 (M+1)⁺); ¹H-NMR (Acetone-d₆, ppm): 3.15 (m, 3H); 3.57 (m, 1H); 3.74 (s, 3H); 4.01 (m, 2H); 4.62 (t, 1H); 6.73 (ddd, 1H); 6.93-6.99 (m, 2H); 7.11-7.18 (m, 2H); 7.28 (d, 1H); 7.62 (td, 1H); 8.52 (m, 1H). ¹³C-NMR (Acetone-d₆, ppm): 34.56; 40.16; 52.18; 55.75; 65.74; 112.86; 115.18; 121.50; 122.74; 124.55; 130.51; 137.52; 146.20; 150.23; 161.03; 163.58; 169.14. Elemental Analysis: calc. C, 68.43%; H, 6.08%; N, 9.39%; found C, 68.28%; H, 6.20%; N, 9.38%.

### Example 29

Z,E 2-[2-(3-methoxy-phenyl)-2-pyridin-4-yl-vinyl]-4,5-dihydro-imidazole-1-carboxylic Acid Tert-Butyl Ester

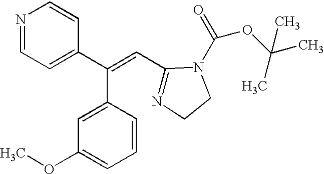

Dry THF (20 mL) was cooled to −78° C. and BuLi (6.15 mL, 9.85 mmol 1.6 N in hexane) was added followed by addition of 2-(diethoxy-phosphorylmethyl)-4,5-dihydro-imidazole-1-carboxylic acid tert-butyl ester (3 g, 9.37 mmol) dissolved in THF (20 mL). The temperature of the reaction mixture was allowed to rise to 10° C. for 5 min. Subsequently it was cooled to −20° C. 4-pyridyl 3-methoxyphenyl ketone (2 g, 9.38 mmol) in THF (10 mL) was added.

Stirring at −20° C. for 20 min followed by stirring at room temperature overnight. NH₄Cl (satd. 25 mL) was added to the reaction mixture resulted in the precipitation of a colourless solid. The mixture was extracted with ethyl acetate (2×50 mL), the organic phases dried over MgSO₄, filtered and evaporated to dryness resulting in a orange oil (4.13 g). This crude mixture was purified on silicagel using dichloromethane /MeOH (19/1) as eluent resulting in an orange oil (3 g, 84%) of a Z,E-mixture of 2-[2-(3-methoxy-phenyl)-2-pyridin-4-yl-vinyl]-4,5-dihydro-imidazole-1-carboxylic acid tert-butyl ester. LCMS: m/z 380 (M+1)⁺; retention time: 2.15 and 2.47 min (ratio 1:2).

### Example 30

7-[2-(4,5-Dihydro-1H-imidazol-2-yl)-1-pyridin-2-yl-ethyl]-2,3-dihydro-benzofuran-5-ol

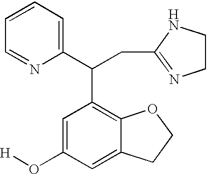

2-[2-(4,5-Dihydro-1H-imidazol-2-yl)-1-(5-methoxy-2,3-dihydro-benzofuran-7-yl)-ethyl]-pyridine (0.21 g; 0.65 mmol) was dissolved in acetonitrile (10 mL). Trimethylsilyl iodide (1 mL) was added and the reaction mixture was heated to reflux for 2 days under nitrogen. After further standing at room temperature for 6 days, Na₂S₂O₄ (5 mL, 1M) was added with the result that the iodine colour disappeared. The reaction mixture was extracted with dichloromethane (2×30 mL) and the combined organic phases dried (MgSO₄) and the solvent removed to give 130 mg of an oil.

Addition of dichloromethane to the oil resulted in precipitation of some crystals which were isolated (85 mg). These crystals were identified as the hydroiodide salt of 7-[2-(4,5-Dihydro-1H-imidazol-2-yl)-1-pyridin-2-yl-ethyl]-2,3-dihydro-benzofuran-5-ol. LCMS: m/z 310 (M+1)⁺; retention time: 1.62 min. Mp: 186-187° C. Calc. for C₁₈H₂₀N₃O₂I; C, 49.57%; H, 4.61%; N, 9.60%; found C, 48.96%; H, 4.57%; N, 9.40%. ¹³C NMR (CDCl₃) ppm:172.3; 161.6; 153.1; 152.4; 150.3; 139.0; 130.4; 125.1; 124.1; 123.8; 114,4; 113,1; 72.9; 46.2; 45.5; 31.8; 31.7. ¹H NMR (CDCl₃) ppm:8.52(d,1H); 7.72(dt,1H); 7.26(m,2H); 6.62(d,1H); 6.33(d,1H); 4.82(s,NH); 4.72(t,1H); 4.52(t,2H); 3.82(s,4H); 3.47(dd,1H); 3.25(dd,1H); 3.13(t,2H).

### Example 31

Separation of the Enantiomers of 2-[2-(4,5-dihydro-1H-imidazol-2-yl)-1-(3-ethoxy-phenyl)-ethyl]-pyridine

A racemic mixture of 2-[2-(4,5-dihydro-1H-imidazol-2-yl)-1-(3-ethoxy-phenyl)-ethyl]-pyridine was separated on a Chiralpak® AD column using 20:80:0,1, 2-propanol:n-heptane:diethylamine as eluent to yield 49 mg (28%) of (+)-2-[2-(4,5-dihydro-1H-imidazol-2-yl)-1-(3-ethoxy-phenyl)-ethyl]-pyridine

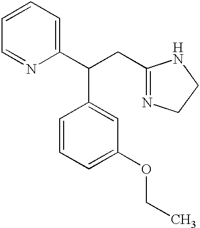

as the 1^{st} eluting isomer: retention time: 9.6 min, ee >99,8%; optical rotation: +27.70, and 56 mg (32%) of (−)-2-[2-(4,5-dihydro-1H-imidazol-2-yl)-1-(3-ethoxy-phenyl)-ethyl]-pyridine

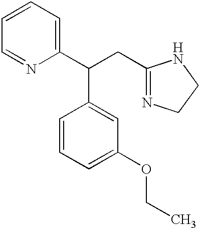

as the 2^{nd} eluting isomer: retention time: 12.9 min, ee >99.3%, optical rotation: −28.20.

### Example 32

2-[2-(4,5-Dihydro-1H-imidazol-2-yl)-1-(3-[1,3]dioxolan-2-yl-phenyl)-vinyl]-pyridine

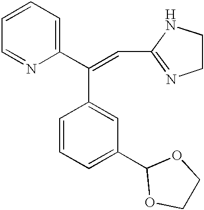

A mixture of 3-bromobenzaldehyde (158.9 g, 0.858 mol) ethylene glycol (55 mL, 1 mol) and TosOH (100 mg) in toluene 150 ml was heated to reflux. Water was collected with a Dean-Stark trap. After 4 h the theoretical am ount of water was collected. The mixture was cooled to room temperature and poured in saturated NaHCO₃ (200 ml). The organic layer was separated washed twice with brine (100 ml), dried over Na₂SO₄ and concentrated. 200 g (>95% yield) of the acetal as an oil was isolated, which was used without purification for the next reaction step.

To a stirred cooled mixture of the above mentioned acetal (45.8 g, 0.2 mol) in THF (250 ml) at −78° C. was added n-BuLi (2.5 M, 80 ml, 0.2 mol) dropwise the mixture turned orange and after some time a precipitate appeared. After 20 min a solution of 2-cyanopyridine (20.8 g, 0.2 mol) in THF 100 ml was added over 10 min. After addition the mixture was allowed to warm over 3 h to 0° C. The mixture was poured in a mixture of acetic acid (35 ml, mol) and ice (100 g). The mixture was stirred for 1 h and NaOH 33% was added to pH ca 11. Toluene (100 ml) was added and the organic layer was separated washed with brine (100 ml), dried over Na₂SO₄ and concentrated. 3-([1,3]dioxolan-2-yl-phenyl)-2-pyridyl ketone was obtained as an almost pure oil (55 g, yield: >90%), this was used without further purification for the next reaction step.

To a stirred cooled mixture of N-BOC 2-methylimidazoline (17.4 g, 0.094 mol) in THF (250 ml) at 5° C. was added in three portions t-BuOK (21.16 g, 0.189 mol). After 5 min a solution of 3-(2-dioxolanylphenyl)-2-pyridyl ketone (22.5 g, 0.09 mol) in THF 100 ml was added over 10 min. After addition the mixture was allowed to warm to room temperature and after some time a solid mass appeared. After 18 h the mixture was poured in a mixture of water/ice (ca 200 ml). After the ice had melted, toluene (100 ml) was added, the organic layer was separated washed with water, dried over Na₂SO₄ and concentrated. 31 g of an oil was obtained, the NMR of this crude product revealed a EIZ=5/1 mixture of 2-[2-(4,5-dihydro-1H-imidazol-2-yl)-1-(3-[1,3]dioxolan-2-yl-phenyl)-vinyl]-pyridine.

m/z 322 (M+1)⁺, HPLC: retention time: 5.0 min, Mp: 98.0-98.5° C. (fumarate).

### Example 33

2-[2-(4,5-Dihydro-1H-imidazol-2-yl)-1-(3-[1,3]dioxolan-2-yl-phenyl)ethyl]pyridine

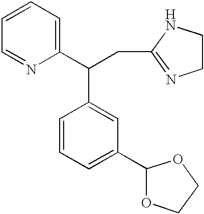

A mixture of Pd/C (10%, 2.2 g), crude 2-[2-(4,5dihydro-1H-imidazol-2-yl)-1-(3-[1,3]dioxolan-2-yl-phenyl)vinyl]pyridin (31 g) and EtOH (250 mL) was hydrogenated at 5 bar at room temperature. After 18 h all starting material was consumed. The mixture was filtered through Celite® and concentrated. Fumaric acid (10.44 g, 0.09 mol) and 2-butanone were added to the concentrate and the mixture was heated to reflux and crystallized. This resulted in 36 g fumarate salt of after filtration.

m/z 324 (M+1)⁺, HPLC: retention time: 4.8 min, Mp: 162.0-162.5° C. (fumarate).

### Example 34

3-[2-(4,5-Dihydro-1H-imidazol-2-yl)-1-pyridin-2-yl-ethyl]-benzaldehyde

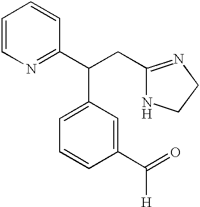

A mixture of 2-[2-(4,5-dihydro-1H-imidazol-2-yl)-1-(3-[1,3]dioxolan-2-yl-phenyl)ethyl]pyridine acetal (18 g, 55 mmol), acetic acid (21 mL) and water (60 mL) was stirred at 40° C. after 4 h the NMR spectrum of a sample revealed >95% conversion.

The aqueous layer was basified with KOH (5%) to pH>1 1 and extracted with dichloromethane (4×40 ml), the organic layer was separated, dried over Na₂SO₄ and concentrated. 13.6 g of a brown oil was obtained, which was precipitated as the fumarate salt. m/z 280 (M+1)⁺, HPLC: retention time: 7.5 min, Mp: 138-139° C. (fumarate).

### Example 35

3-[2-(4,5-Dihydro-1H-imidazol-2-yl)-1-pyridin-2-yl-ethyl]-phenyl-methanol

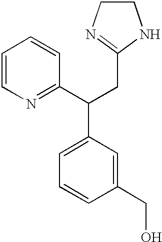

To a stirred solution of the aldehyde (Example 34) (5.0 g, 18 mmol) in EtOH (75 mL) at 5° C. was added NaBH₄ (0.486 g, 13.5 mmol) in 2 portions over 15 min. After 18 h at room temperature, acetic acid (3 mL) was added after 15 min. the mixture was concentrated at the rotavapor. CH₂Cl₂ (50 ml) and water (50 mL) were added KOH (5%) was added to pH 11 the organic layer was separated, dried over Na₂SO₄ and concentrated. Ca 4.8 g of a brown oil was obtained, which was ca 95% pure by NMR. Purification by the fumarate salt crystallization from 2-propanol gave 3.2 g of a white salt together with an oily substance. The salt was dried overnight at room temperature and at 70° C. for 1 h resulting in a hard oil containing one mol equivalent 2-propanol. m/z 282 (M+1)⁺, HPLC: retention time: 3.7 min. Mp: 114-115° C. (fumarate).

### Example 36

Z/E-3-(3-Methoxy-phenyl)-2-methyl-3-pyridin-3-yl-acrylonitrile

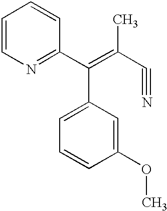

Dry THF (20 mL) was coled to −78° C., BuLi (5.5 mL, 1.6 N in hexane) was added followed by addition of diethyl 1-cyanoethylphosphonate (1.6 g, 8.4 mmol) dissolved in THF (20 mL).The temperature was allowed to rise to 10° C. for 5 min, then cooled to −20° C. followed by addition of 2-pyridyl 3-methoxyphenyl ketone (1.8 g, 8.4 mmol) in THF (10 mL). Stirring at −20° C. for 20 min followed by stirring at room temperature overnight. NH₄Cl (satd. 25 mL) was added to the reaction mixture resulted in the precipitation of a colourless solid. The mixture was extracted with EtOAc (2×50 mL), the organic phases dried over MgSO₄, filtered and evaporated to dryness resulting in a greenish oil (2.0 g, 95%). LCMS: m/z 251 (M+1)⁺; retention time: 3.17 and 3.51min, respectively. ¹H NMR (CDCl₃) ppm: 8.68(dd,1H); 7.68(dd,1H); 7.48-7.04(m,3H); 6.96(dt,2H); 6.72(dd, 1H);3.78(s,3H); 2.14 and 2.08(s, ratio 7/5,3H).

### Example 37

2-[2-(4,5-Dihydro-1H-imidazol-2-yl)-1-(3-methoxy-phenyl)-propyl]-pyridine

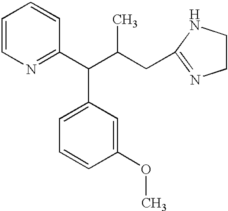

Z/E-3-(3-Methoxy-phenyl)-2-methyl-3-pyridin-3-yl-acrylonitrile (1,2 g, 4.8 mmol) was reacted with 1,2-ethylenediamine monotosylate (3.4 g, 14.4 mmol) by heating to 160° C. for 48 h.

NaOH (20 mL, 1N) and CHCl₃ (80 mL) was added, the organic layer separated, dried with MgSO₄ and evaporated to dryness. The crude product was purified on silicagel using ethyl acetate/ethanol/trifluoroacetic acid (4:4:1) as eluent. 325 mg of 2-[2-(4,5-dihydro-1H-imidazol-2-yl)-1-(3-methoxy-phenyl)-propyl]-pyridine was isolated as an oil. LCMS: retention time: 1.45 and 1.62; m/z 296 (M+1)⁺.

### Example 38

2-[2-quinolin-2-yl-2-(3-trifluoromethyl-phenyl)-vinyl]-4,5-dihydro-imidazole-1-carboxylic Acid Tert-Butyl Ester

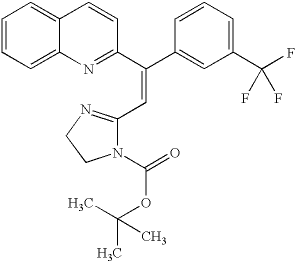

2-Quinolinyl 3-trifluoromethylphenyl ketone (2.0 g, 6.6.4 mmol) was treated with 2-(diethoxyphosphorylmethyl)-4,5-dihydro-imidazole-1-carboxylic acid tert-butyl ester (2.12 g, 6.64 mmol) in THF (5.0 mL) with Bu Li ((5.0 mL, 1.6 N in hexane) as described above.Purification of the crude product was performed on Flash 40 using EtOAc/heptane (4/3) as eluent. Yield: 1.4 g (45%) of 2-[2-quinolin-2-yl-2-(3-t rifluoromethyl-phenyl)-vinyl]-4,5-dihydro-imidazole-1-carboxylic acid tert-butyl ester. m/z (M+1)⁺468; ELS-purity: 100%.

2-[2-(4,5-Dihydro-1H-imidazol-2-yl)-1-(3-trifluoromethyl-phenyl)-vinyl]-quinoline

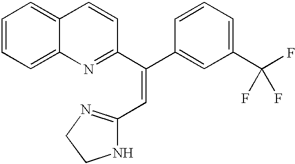

0.75 g of 2-[2-quinolin-2-yl-2-(3-trifluoromethyl-phenyl)-vinyl]-4,5-dihydro-imidazole-1-carboxylic acid tert-butyl ester was deprotected by means of trifluoroacetic acid (2.0 mL) in dichloromethane (4.0 mL) by stirring at room temperature for 2 h.

pH was adjusted to >9 with NaOH (10 N), subsequently the mixture was extracted with dichloromethane 3×10 mL, the dichloromethane phases dried over MgSO₄ evaporated to dryness to give 0.53 g (90) of 2-[2-(4,5-dihydro-1H-imidazol-2-yl)-1-(3-trifluoromethyl-phenyl)-vinyl]-quinoline as a solid. mlz 368 (M+1)⁺, retention time: 2.94min, ELS purity: 100%. Mp: 76.5° C.

2-[2-(4,5-Dihydro-1H-imidazol-2-yl)-1-(3-trifluoromethyl-phenyl)-ethyl]-quinoline

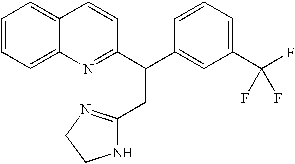

2-[2-(4,5-dihydro-1H-imidazol-2-yl)-1-(3-trifluoromethyl-phenyl)-vinyl]-quinoline (250 mg) was hydrogenated over Pd/C (10%, 25 mg) in EtOH (5 mL) at 30 psi for 48 h.The mixture was filtered through filter aid evaporation to dryness gave 245 mg (99%) of solid 2-[2-(4,5-dihydro-1H-imidazol-2-yl)-1-(3-trifluoromethyl-phenyl)-ethyl]-quinoline. m/z 370 (M+1)⁺, retention time: 2.74min, purity: 100%. Mp: 92-93° C.

### Example 39

3-[2-(4,5-Dihydro-1H-imidazol-2-yl)-1-pyridin-2-yl-ethyl]-phenylamine

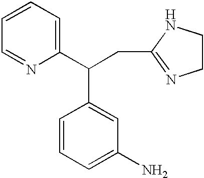

To a stirred mixture of cyanopyridine (20.8 g, 0.2 mol) in THF (120 ml) at 5° C. was added a solution of 3-bis-trimethylsilylamino phenylmagnesium chloride (0.1 M, 200 ml, 0.2 mol) in THF dropwise the mixture turned dark brown. After addition the mixture was 27° C. The mixture was warmed to 40° C. After 3 h the mixture was poured in a mixture of acetic acid (40 ml) and ice (200 g). The mixture was stirred for 1 h and NaOH 33% was added to a pH of ca 11. Toluene (100 ml) was added and the organic layer was separated washed with brine (100 ml), dried over Na₂SO₄ and concentrated. 3-Bistrimethylsilylaminophenyl 2-pyridyl ketone was obtained as an almost pure oil (52 g, yield: >90%). The TMS protection groups were only partly hydrolysed under these reaction conditions. The obtained oil was used as such for the next reaction step.

To a stirred cooled mixture of diethyl cyanomethyl phosphonate (19.5 g, 0.11 mol) in THF (400 ml) at 10° C. was added in two portions t-BuOK (14 g, 0.125 mol). After 5 min a solution of the ketone mentioned above (33 g, 0.1 mol) in THF (150 ml) was added over 10 min. After addition the mixture was allowed to warm to room temperature. After 4 h the mixture was poured in a mixture of water/ice (300 mL). After the ice had melted, toluene (100 mL) was added, the organic layer was separated, washed with water, dried over Na₂SO₄ and concentrated to give an oil. The NMR spectrum of this crude product revealed an E/Z=312 mixture of 3-(3-aminophenyl)-3-pyridin-2-yl-acrylonitrile which was hydrogenated over Pd/C 10%. Pd/C (10%, 3 g ) mixed with the E/Z-mixture of 3-(3-aminophenyl)-3-pyridin-2-yl-acrylonitrile (30 g, 0.13 mol) and EtOH (600 mL) was hydrogenated at 5 bar and 50° C. After 3 h one isomer was consumed and after 18 h also the other isomer was consumed. The mixture was filtered through filter aid and concentrated. Purification on a silicagel column using CH₂Cl₂/MeOH (100/1) as eluent gave 13 g of 3-(3-amino-phenyl)-3-pyridin-2-yl-propionitrile. MS: m/z 224 (M+1)⁺.

A mixture of crude 3-(3-amino-phenyl)-3-pyridin-2-yl-propionitrile (30 g, 130 mmol) and ethylenediamine monotosylate (102 g, 440 mmol) was heated with stirring to 150° C. After 2 h, an NMR spectrum revealed full conversion of starting nitrile. The mixture was cooled to room temperature and KOH (5%, 500 mL) was added. After all the salt was dissolved, the mixture was extracted with CHCl₃, 2×200 mL. The organic layer was dried and concentrated. 16 g of a reasonably pure solid (by NMR) was obtained which was combined with fumaric acid (7.5 g) this salt was dissolved in water (100 ml) and filtered through Celite®. The solution was made basic (pH 10) with KOH (5%). The suspension was extracted with CHCl₃ 2×200 ml. The organic layer was dried and concentrated to give 13 g of a solid which was reprecipitated as the fumarate. MS: m/z 267 (M+1)⁺, HPLC: retention time: 6.3 min. Mp: 154-155° C. (fumarate).

### Example 40

1-(2,3-Dihydro-7-benzofuranyl)-2-(4,5-dihydro-2-imidazolyl)-1-(2-pyridyl)-ethane

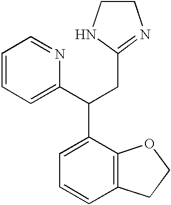

To a stirred cooled mixture of 2,3-dihydrobenzofuran (75 g, 0.625 mol) in diethyl ether (1.8 l) and N,N,N′,N′-tetramethylethylenediamine (95 mL) at −10° C. was added n-BuLi (2.5 M, 250 ml, 0.625 mol) dropwise the mixture turned yellow and after some time a precipitate appeared. After 3.5 h the mixture was cooled to −78° C. and a solution of 2-cyanopyridine (65 g, 0.625 mol) in THF (200 mL) was added over 15 min. After addition the mixture was allowed to warm over 2 h to room temperature. The mixture was poured in a mixture of HCl (10 N, 300 mL) and ice (400 g). The mixture was stirred for 1 h and NaOH 33% was added to a pH of ca 11. The ether was evaporated and the resulting mixture was extracted with dichloromethane 2×200 m L. The organic layer was separated, dried over Na₂SO₄ and concentrated to give an oil. EtOAc 200 ml was added and to this mixture heptane (100 mL) was added dropwise under stirring. The solid formed was filtered and dried to give 67.6 g (88%) of 2,3-dihydrobenzofuran-7-yl 2-pyridyl ketone.

Two portions t-BuOK (8.25 g, 74 mmol) were added to a stirred cooled mixture of diethyl cyanomethylphosphonate (12 g, 67 mmol) in THF (250 mL) at 0° C. After 5 min a solution of 2,3-dihydrobenzofuran-7-yl 2-pyridyl ketone (15.5 g, 66 mmol) in THF (150 mL) was added over 10 min. After addition the mixture was allowed to warm to room temperature. After 18 h the mixture was poured in a mixture of water/ice (300 mL). After the ice had melted, dichloromethane (100 mL) was added, the organic layer was separated washed with water, dried over Na₂SO₄ and concentrated to give an oil. The NMR spectrum of this crude product revealed that an E/Z mixture (20 g) of 3-2,3-dihydrobenzofuran-7-yl-3-(2-pyridyl) acrylonitrile which was reduced without further purification.

A mixture of Pd/C (10%, 2.2 g), crude 3-(2,3-dihydrobenzofuran-7-yl)-3-(2-pyridyl) acrylonitrile (20 g, 0.13 mol) and EtOH (600 mL) was hydrogenated at 5 bar and 50° C. for 18 h, and subsequently the mixture was filtered through Celite® and concentrated. Purification on silicagel using CH₂Cl₂/MeOH (100/1) as eluent gave 8 g of 3-(2,3-dihydrobenzofuran-7-y)-13-(2-pyridyl) propionitrile.

This nitrile was treated with 1,2-ethylenediamine monotosylate (20 g, 85 mmol) by heating with stirring to 140° C. for 2 h The mixture was cooled to room temperature and KOH (5%, 200 mmol) was added after all the salt was dissolved the mixture was extracted with CHCl₃ (2×100 mL). The organic layer was dried and concentrated to give 9 g of an oil which was purified further by bulb-to-bulb distillation (220° C., 0.2 mm Hg). 2 g of the resulting oil was treated with 1 equivalent of fumaric acid in 2-propanol to give 2 g 1-(2,3-dihydro-7-benzofuranyl)-2-(4,5-dihydro-2-imidazolyl)-1-(2-pyridyl)-ethane fumarate. m/z 294 (M+1)⁺; retention time: 6.5 min.

Mp: 166-167° C. (fumarate).

### Example 41

7-[2-(4,5-Dihydro-1H-imidazol-2-yl)-1-pyridin-2-yl-ethyl]-2,3-dihydro-benzofuran-5-ylamine

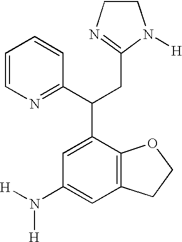

To stirred cooled H₂SO₄ (140 ml) at 10° C. was added in portions 2,3-dihydrobenzofuran-7-yl 2-pyridyl ketone (16 g, 71.1 mmol), the mixture was cooled to −25° C. and a mixture of H₂SO₄ (3.6 ml) and HNO₃ (7.2 g) was added over 20 min. The mixture was allowed to warm over 3 h to room temperature. The mixture was poured on ice (400 g) water (1 L) and NaOH (33%) were added carefully to a pH of 9. The mixture was extracted with chloroform (2×200 mL). The organic layer was separated, dried over Na₂SO₄ and concentrated to give 5-nitro-2,3-dihydrobenzofuran-7-yl 2-pyridyl ketone as a solid (17 g, 88%), which was used as such for the next reaction step.

To a stirred cooled mixture of diethyl cyanomethylphosphonate (12 g, 67 mmol) in THF (250 mL) at 0° C. was added in two portions t-BuOK (8.25 g, 74 mmol). After 5 min a solution of 5-nitro-2,3-dihydrobenzofuran-7-yl 2-pyridyl ketone (18 g, 66.6 mmol) in THF (150 mL) was added over 10 min. After the addition, the m ixture was allowed to warm to room temperature. After 18 h, the mixture was poured in a mixture of water/ice (300 ml). After the ice had melted, dichloromethane (100 mL) was added, the organic layer was separated, washed with water, dried over Na₂SO₄ and concentrated to give an E/Z mixture (22 g) of 3-(5-nitro-2,3-dihydrobenzofuran-7-yl)-3-(2-pyridyl)-acrylonitrile.

This nitrile was hydrogenated over Pd/C (10%, 2.2 g) in EtOH (300 mL) at 5 bar and room temperature. After 3 h, the nitro functionality was reduced. The reduction of the alkene functionality was carried out for further 18 h at 50° C. The mixture was filtered through Celite® and concentrated to give 3-(5-amino-2,3-dihydrobenzofuran-7-yl)-3-(2-pyridyl)-propionitrile (18 g) as a solid.

11.5 g (43.3 mmol) of this was treated with 1,2-ethylenediamine monotosylate (40 g, 173 mmol) by heating with stirring to 140° C., after 1 h the mixture was cooled to room temperature and KOH (5%, 200 mmol) was added. After all the salt was dissolved the mixture was extracted with CHCl₃ (2×100 mL). The organic layer was dried and concentrated to give 12 g of a reasonably pure solid which was recrystallized from EtOAc. Yield: 7.5 g of 7-[2-(4,5-dihydro-1H-imidazol-2-yl)-1-pyridin-2-yl-ethyl]-2,3-dihydro-benzofuran-5-ylamine. m/z 309 (M+1)+; retention time: 6.9 min, Mp: 143-144° C.

### Example 42

Separation of the Enantiomers of 2-[2-(4,5-Dihydro-1H-imidazol-2-yl)-1-(5-bromo-2,3-dihydrobenzofuran-7-yl)-ethyl]-pyridine

2-[2-(4,5-Dihydro-1H-imidazol-2-yl)-1-(5-bromo-2,3-dihydro-benzofuran-7-y l)-ethyl]-pyridine was separated in enantiomers on a Chiralpak® AD column, using 2-propanol:heptane:diethylamine, 20:80:0.1 as eluent.

(+)-2-[2-(4,5-Dihydro-1H-imidazol-2-yl)-1-(5-bromo-2, 3-dihydro-benzofuran-7-yl)-ethyl]-pyridine

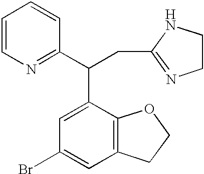

Yield: 43%, retention time: 11.1 min. Purity: >99,8% ee. Optical rotation: +220, (2-propanol/heptane/diethylamine, 20/80/0.1), 25° C.

(−)-2-[2-(4,5-Dihydro-1H-imidazol-2-yl)-1-(5-bromo-2,3-dihydro-benzofuran-7-yl)-ethyl]-pyridine

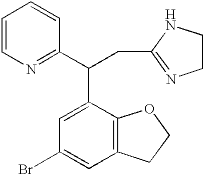

Yield: 42%, retention time: 13.8 min. purity: >99,4% ee. Optical rotation: -27.40 (2-propanol/heptane/diethylamine, 20/80/0.1), 25° C.

### Example 43

Isobutyric Acid {3-[2-(4,5-dihydro-1H-imidazol-2-yl)-1-pyridin-2-yl-ethyl]-benzylidene}-hydrazide

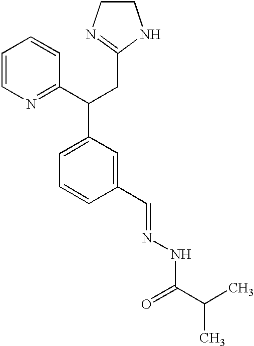

3-[2-(4,5-Dihydro-1H-imidazol-2-yl)-1-pyridin-2-yl-ethyl]-benzaldehyde (80 mg,286 mmol) was dissolved in ETOH (2 mL), isobutyric acid hydrazide (35 mg, 340 mmol) was added and the mixture stirred at room temperature for 2 days. The solution was evaporated to an oil which was dissolved in dichloromethane (5 mL) treated with PS-TsNHNH₂-scavenging resin for 1 h, filtered, the residue was washed with dichloromethane (5×2 mL) and the combined filtrates were evaporated to dryness to give an oil. Yield: 75 mg (70%). mlz 365 (M+1)⁺. Retention time: 1.6 min, ELS purity: 100%.

### Example 44

{4-[2-(4,5-Dihydro-1H-imidazol-2-yl)-1-pyridin-2-yl-ethyl]-2,3-dihydro-benzofuran-6-yl}-pentylamine

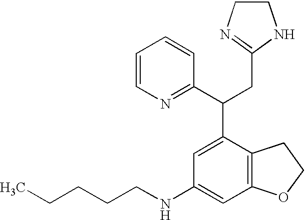

Pentanal (182 m g, 2.12 mmol) and NaCNBH₃ (132 mg, 2.12 mmol) were mixed in MeOH (20 mL). 7-[2-(4,5-Dihydro-1H-imidazol-2-yl)-1-pyridin-2-yl-ethyl]-2,3-dihydro-benzofuran-5-ylamine (493. mg, 1.6 mmol) was added slowly followed by stirring the mixture overnight in a N₂ atmosphere. The mixture was evaporated to a brownish oil which was purified on silicagel on Flash 40. Yield: 400 mg of {4-[2-(4,5-Dihydro-1H-imidazol-2-yl)-1-pyridin-2-yl-ethyl]-2,3-dihydro-benzofuran-6-yl}pentylamine (78%).

m/z: 379 (M+1)⁺, retention time: 1.6 min, ELS purity; 89% (22% as Na-salt).

### Example 45

{4-[2-(4,5-Dihydro-1H-imidazol-2-yl)-1-pyridin-2-yl-ethyl]-2,3-dihydro-benzofuran-6-yl}-heptylamine

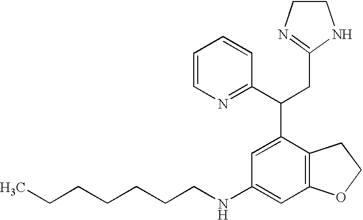

Heptanal (240 mg, 2.12 mmol) and NaCNBH₃ (132 mg, 2.12 mmol) were mixed in MeOH (20 mL). 7-[2-(4,5-Dihydro-1H-imidazol-2-yl)-1-pyridin-2-yl-ethyl]-2,3-dihydrobenzofuran-5-ylamine (493 mg, 1.6 mmol) was added slowly followed by stirring the mixture overnight in a N₂ atmosphere. The mixture was evaporated to a brownish oil which was purified on silicagel on Flash 40. Yield: 405 mg of {4-[2-(4,5-dihydro-1H-imidazol-2-yl)-1-pyridin-2-yl-ethyl]-2,3-dihydro-benzofuran-6-yl}heptylamine (60%)

m/z: 407 (M+1)⁺, retention time: 2.39 min.

### Example 46

2-[1-(5-Bromo-2,3-dihydrobenzofuran-7-yl)-2-(4,5-dihydro-1H-imidazol-2-yl)-ethyl]-3-methylpyridine

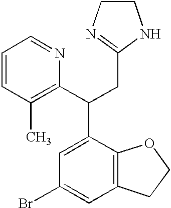

A solution of Br₂ (1.6 mmol, 256 mg) in dichloromethane (2 mL) was dropwise added to a mixture of 2-[1-(2,3-dihydro-benzofuran-7-y l)-2-(4,5-dihydro-1H-imidazol-2-yl)-ethyl]-3-methyl-pyridine, (1.6 mmol, 500 mg) and acetic acid (25 mL) under nitrogen. After stirring overnight, the solvent was evaporated. The solid residue was extracted between NaOH 1M (75 mL) and dichloromethane (3×50 mL). The combined organic fractions were dried over MgSO₄, filtered, and evaporated to dryness to give pale yellowish crystals, (490 mg, 79%). LC-MS retention time: 2.31 min, m/z: 387 (M+1)⁺. ¹H-NMR (CDCl₃, ppm): 2.21 (s, 3H); 2.83 (dd, 1H); 3.14 (t, 2H); 3.28 (dd, 1H); 3.40 (m, 4H); 4.56 (t, 2H); 5.10 (s, broad; 1H:NH); 6.92 (d, 1H); 7.01-7.06 (m, 2H); 7.34 (dd, 1H); 8.40 (dd, 1H). ¹³C-NMR (CDCl3, ppm): 18.90; 30.25; 33.96; 40.39; 50.13; 71.76; 112.57; 122.08; 126.34; 126.73; 129.29; 129.58; 132.55; 138.39; 146.48; 157.07; 159.36; 167.49.

### Example 47

2-[2-(4,5-Dihydro-1H-imidazol-2-yl)-1-(5-iodo-2,3-dihydro-benzofuran-7-yl)-ethyl]-3-methylpyridine

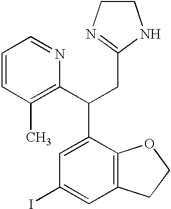

A solution of ICI (3.3 mmol, 532 mg) in acetic acid (5 mL) was dropwise added to a mixture of 2-[1-(2,3-dihydro-benzofuran-7-yl)-2-(4,5-dihydro-1H-imidazol-2-yl)-ethyl]-3-methyl-pyridine (1.64 mmol, 504 mg) and acetic acid (5 mL) in inert atmosphere (N₂). After stirring overnight, NaS₂O₃ (1M, 3 mL) was added until the mixture became orange. The solvent was evaporated and the resulting residue was extracted between NaOH (1M, 50 mL) and dichloromethane (4×25 mL). The combined organic fractions were dried over MgSO₄, filtered and evaporated to dryness to give the crude product. Purification by Flash 40 silica column chromatography using EtOAc/EtOH/triethylamine (4:4:1) as eluent gave the product (150 mg, 21%). Decomposition inside the column was observed. LC-MS: retention time: 2.43 min, m/z: 434 (M+1)+. ¹H-NMR (CDCl₃, ppm): 2.20 (s, 3H); 2.85 (dd, 1H); 3.18 (t, 2H); 3.30 (dd, 1H); 3.44 (m, 4H); 4.58 (t. 2H); 4.85 (dd, 1H); 5.47 (s, broad; 1H: NH); 7.03-7.10 (m, 2H); 7.28-7.40 (m, 2H); 8.42 (dd, 1H). ¹³C-NMR (CDCl3, ppm): 18.94; 30.11; 40.31; 40.42; 49.49; 71.79; 82.41; 122.28; 126.95; 130.03; 132.40; 132.76; 135.51; 138.62; 146.45; 157.99; 159.12; 166.58; 168.08.

### Example 48

2-[2-(5-Bromo-2,3-dihydro-benzofuran-7-y l)-2-pyridin-2-yl-ethyl]-4,5-dihydro-imidazole-1-carboxylic Acid Tert-Butyl Ester

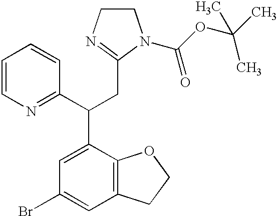

A mixture of 2-[1-(5-bromo-2,3-dihydro-benzofuran-7-yl)-2-(4,5-dihydro-1H-imidazol-2-yl)-ethyl]-pyridine (7.41 mmol, 2.76 g), dry dichloromethane (30 mL), triethylamine (11.3 mmol, 1.57 mL), and BOC₂O (11.3 mmol, 11.3 mL) was stirred in N₂ atmosphere for 16 h. The solvent was evaporated and the resulting oil dissolved in dichloromethane (50 mL). The organic fraction was washed with NaOH (0.1M, 40 mL), H₂O (40 mL) and brine (10 mL), dried over MgSO₄, filtered and evaporated. The crude oil was purified by Flash 40 silica column chromatography using dichloromethane/MeOH (19:1) to give 780 mg (22%). ¹H-NMR (CDCl₃, ppm): 1.49 (s, 9H); 3.13 (t, 2H); 3.30 (dd; 1H); 3.52-3.68 (m, 4H); 3.78 (dd, 1H); 4.52 (t, 2H); 4.88 (dd, 1H); 7.02-7.09 (m, 2H); 7.15 (d, 1H); 7.24 (d, 1H); 7.53 (ddd, 1H); 8.51 (ddd, 1H). ¹³C-NMR (CDCl3, ppm): 28.19; 29.83; 34.32; 43.43; 46.64; 51.99; 71.29; 81.60; 112.18; 121.33; 123.38; 125.93; 126.98; 128.94; 129.72; 136.20; 148.94; 150.92; 156.91; 159.31; 161.58.

2-[2-Pyridin-2-yl-2-(5-p-tolyl-2,3-dihydro-benzofuran-7-yl)-ethyl]-4,5-dihydro-imidazole-1-carboxylic Acid Tert-Butyl Ester

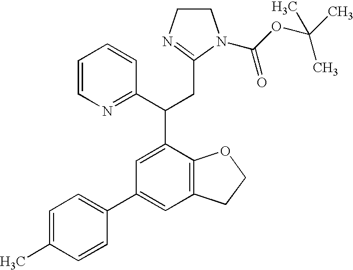

A solution of 2-[2-(5-bromo-2,3-dihydro-benzofuran-7-yl)-2-pyridin-2-yl-ethyl]-4,5-dihydroimidazole-1-carboxylic acid tert-butyl ester (0.82 mmol, 390 mg), 4-methyl benzenboronic acid (0.82 mmol, 112 mg) and K₂CO₃ (2M, 1.6 mL) in toluene (16 mL) and EtOH (1.6 mL) was degassed by bubbling N₂ through the solution for 30 min, before adding Pd(PPh₃)₄ (4 mol %, 38 mg). After three cycles of evacuation and re-filling with N₂, the mixture was heated to 80° C. After 2 h, LC-MS showed 92% conversion. The reaction mixture was cooled to room temperature, quenched with satd. NH₄Cl (10 mL) and extracted with EtOAc (3×10 mL). The combined organic fractions were dried over MgSO₄, filtered and evaporated. Purification by Flash 40 silica column chromatography, using dichloromethane/MeOH (19:1) as eluent, gave (200 mg). Complete purification was not carried out due to decomposition of the compound.

2-[2-(4,5-Dihydro-1H-imidazol-2-yl)-1-(5-p-tolyl-2,3-dihydro-benzofuran-7-yl)-ethyl]-pyridine

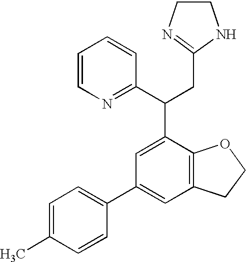

A mixture of 2-[2-pyridin-2-yl-2-(5-p-tolyl-2,3-dihydro-benzofuran-7-yl)-ethyl]-4,5-dihydroimidazole-1-carboxylic acid tert-butyl ester (0.37 mmol, 180 mg), trifluoroacetic acid (8 mL), and dichloromethane (4 mL) was stirred at room temperature during 2 h. pH was adjusted to 10 using NaOH 4M. The resulting mixture was extracted with dichloromethane (2×50 mL). The combined organic fractions were dried over MgSO₄, filtered and evaporated. The raw product was purified by semi preparative HPLC to give 41 mg (29%). LC-MS: retention time: 2.68 min, m/z: 384 (M+1)⁺. ¹H-NMR (CDCl₃, ppm): 2.35 (s, 3H); 3.00 (dd, 1H); 3.22 (t, 2H); 3.39 (dd, 1H); 3.43 (s, broad; 4H); 4.59 (t, 2H); 4.77 (dd, 1H); 7.06-7.37 (m, 8H); 7.54 (ddd, 1H); 8.54 (ddd, 1H). ¹³C-NMR (CDCl3, ppm): 21.01; 30.04; 33.14; 44.74; 49.77 (br); 71.39; 121.55; 122.00; 123.67; 124.92; 125.75; 126.63; 127.58; 129.32; 134.09; 136.14; 136.51; 138.45; 148.80; 157.05; 161.88; 167.21.

### Example 49

2-[1-(5-Bromo-2,3-dihydro-benzofuran-7-yl)-2-(4,5-dihydro-1H-imidazol-2-yl)-ethyl]-pyridine

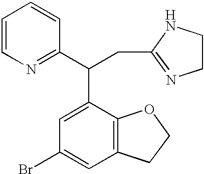

A solution of Br₂ (11.4 mmol, 0.582 mL) was dropwise added to a mixture of 1-(2,3-dihydro-7-benzofuranyl)-2-(4,5-dihydro-2-imidazolyl)-1-(2-pyridyl)-ethane (11.4 mmol, 3.34 g) and AcOH (75 mL) in inert atmosphere (N₂). After stirring overnight, the solvent was evaporated. The solid residue was extracted between NaOH 1 M (50m L) and dichloromethane (3×50 mL). The combined organic fractions were dried over MgSO₄, filtered and the solvent evaporated to dryness to give brownish crystals (3.96 g, 93%). LC-MS: retention time: 1.90 min, m/z: 373/374 (M+1)⁺. ¹H-NMR (CDCl₃, ppm): 2.80 (dd, 1H); 3.00 (t, 2H); 3.20 (dd, 1H); 3.32 (m, 4H); 4.39 (t, 2H); 4.62 (dd, 1H); 5.78 (s, broad; 1H:NH); 6.94-7.00 (m, 3H); 7.12 (d, 1H); 7.42 (t, 1H); 8.39 (d, 1H). ¹³C-NMR (CDCl₃, ppm): 30.08; 32.95; 44.38; 49.54; 49.68; 71.75; 112.47; 122.06; 123.85; 126.51; 126.78; 129.59; 129.65; 136.88; 149.19; 157.07; 161.24; 167.30.

### Example 50

Separation of the Enantiom ers of 2-[2-(4,5-dihydro-1H-imidazol-2-yl)-1-(5-methyl-2,3-dihydrobenzofuran-7-yl)-ethyl]-pyridine

Racemic 2-[2-(4,5-dihydro-1H-imidazol-2-yl)-1-(5-methyl-2,3-dihydro-benzofuran-7-yl)-ethyl]-pyridine was separated on a Chiralcel® OD, column using 10:90:0,1, 2-propanol:n-heptane:diethylamine as eluent.

Yield: 40% of the 1^{st} eluting isomer of 2-[2-(4,5-dihydro-1H-imidazol-2-yl)-1-(5-methyl-2,3-dihydro-benzofuran-7-y l)-ethyl]-pyridine pyridine,

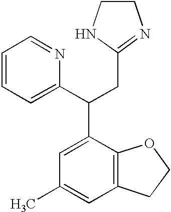

retention time: 14.8 min, >98% ee;

and 24% of the 2^{nd}. eluting isomer of 2-[2-(4,5-dihydro-1H-imidazol-2-yl)-1-(5-methyl-2,3-dihydro-benzofuran-7-yl)-ethyl]-pyridine pyridine.

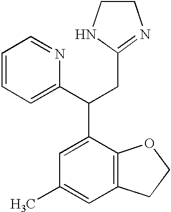

### Example 51

Separation of the Enantiomers of 2-[1-(2,3-dihydro-benzofuran-5-yl)-2-(4,5-dihydro-1H-imidazol-2-yl)-ethyl]-pyridine

Racemic 2-[i -(2,3-dihydro-benzofuran-5-yl)-2-(4,5-dihydro-1H-imidazol-2-yl)-ethyl]-pyridine was separated on a Chiralpak® AD column using 50:50:0,1, 2-propanol:n-heptane:diethylamine as eluent.

Yield of 1^{st} eluting isomer: 43% of (+)-2-[1-(2,3-dihydro-benzofuran-5-yl)-2-(4,5-dihydro-1H-imidazol-2-yl)-ethyl]-pyridine,

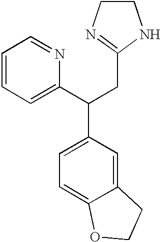

retention time: 11.8 min, 93.1% ee, optical rotation: +70,1°, 50:50:0,1, 2-propanol:n-heptane: diethylamine.

Yield of 2^{nd} eluting isomer: 34% of (−)-2-[1-(2,3-dihydro-benzofuran-5-yl)-2-(4,5-dihydro-1H-imidazol-2-yl)-ethyl]-pyridine,

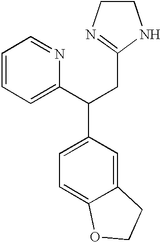

retention time: 14.5 min 99.2% ee, optical rotation: +70,5°, 50:50:0,1, 2-propanol:n-heptane:diethylamine

### Example 52

Separation of the Enantiomers of 3-[2-(4,5-dihydro-1H-imidazol-2-yl)-1-phenyl-ethyl]-5,6,7,8-tetrahydro-imidazol[1,5,-a]pyridine

Racemic 3-[2-(4,5-dihydro-1H-imidazol-2-yl)-1-phenyl-ethyl]-5,6,7,8-tetrahydro-imidazo[1,5-a]pyridine was separated on a Chiralpak® AD column using 50:50:0,1, 2-propanol:n-heptane:diethylamine as eluent.

Yield of 1^{st} eluting isomer: 44% of (−)-3-[2-(4,5-dihydro-1H-imidazol-2-yl)-1-pheyl-ethyl]-5,6,7,8-tetrahydro-imidazo[1,5-a]pyridine

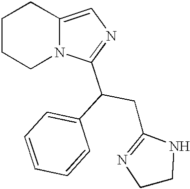

Retention time: 9.1 min: 99.3% ee. Optical rotation: −160.4°, 50:50:0, 1, 2-prpanol:n heptane:diethylamine

Yield of 2^{nd} eluting isomer: 42% of (+)-3-[2-(4,5-dihydro-1H-imidazol-2-yl)-1-phenyl-ethyl]-5,6,7,8-tetrahydro-imidazo[1,5-a]pyridine.

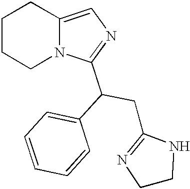

Retention time: 16.2 min: 99.5% ee. Optical rotation: +169.90, 50:50:0,1, 2-propanol:n-heptane:diethylamine

### Example 53

Separation of the Enantiomers of 2-[1-(5-tert-butyl-2,3-dihydro-benzofuran-7-yl)-2-(4,5-dihydro-1H-imidazol-2-yl)ethyl]pyridine

Racemic 2-[1-(5-tert-butyl-2,3-dihydro-benzofuran-7-yl)-2-(4,5-dihydro-1H-imidazol-2-yl)-ethyl]pyridine was separated on a Chiralpak® AD column using 90:10:0,1, heptane: 2-propanol:diethylamine as eluent.

Yield of 1^{st} eluting isomer: 46% of (−)-2-[1-(5-tert-butyl-2,3-dihydro-benzofuran-7-yl)-2-(4,5-dihydro-1H-imidazol-2-yl)-ethyl]-pyridine,

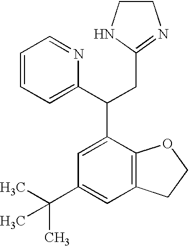

retention time: 9.2 min. >99.5% ee, optical rotation: −42.20.

Yield of 2^{nd}. eluting isomer: 45% of (+)-2-[1-(5-tert-butyl-2,3-dihydro-benzofuran-7-yl)-2-(4,5-dihydro-1H-imidazol-2-yl)-ethyl]-pyridine, retention time: 11.7 min. >99.5% ee, optical rotation: +47°.

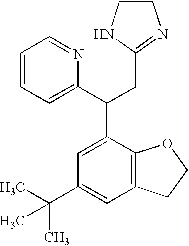

### Example 54

{3-[2-(4,5-Dihydro-1H-imidazol-2-yl)-1-pyridin-2-yl-ethyl]phenyl}pentylamine

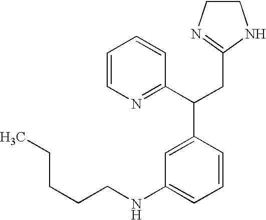

Pentanal (97 mg,1.13 mmol) and NaCNBH₃ (70.7 mg,1.13 mmol) were mixed in MeOH (10 mL). 3-[2-(4,5-dihydro-1H-imidazol-2-yl)-1-pyridin-2-yl-ethyl]phenylamine (200 mg, 0.75 mmol) was added slowly followed by stirring the mixture overnight in a N₂ atmosphere. The mixture was evaporated to a brownish oil which was purified on silicagel on Flash 40.using EtOAc:EtOH: triethylamine, 4:4:1 as eluent. Yield: 110 mg (45%) of {3-[2-(4,5-dihydro-1H-imidazol-2-yl)-1-pyridin-2-yl-ethyl]-phenyl}pentylamine m/z: 337 (M+1)+, retention time: 2.33, ELS-purity: 95%.

### Example 55

{3-[2-(4,5-Dihydro-1H-imidazol-2-yl)-1-pyridin-2-yl-ethyl]phenyl}heptylamine

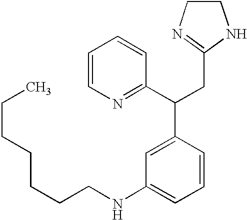

Heptanal (129 mg,1.13 mmol) and NaCNBH₃ (70.7 mg,1.13 mmol) were mixed in MeOH (10 mL). 3-[2-(4,5-dihydro-1H-imidazol-2-yl)-1-pyridin-2-yl-ethyl]phenylamine (200 mg, 0.75 mmol) was added slowly followed by stirring the mixture overnight in a N₂ atmosphere. The mixture was evaporated to a brownish oil which was purified on silicagel on Flash 40.using EtOAc/EtOH/triethylamine 4/4/1 as eluent. Yield: 120 mg (45%) of {3-[2-(4,5-dihydro-1H-imidazol-2-yl)-1-pyridin-2-yl-ethyl]phenyl}heptylamine m/z: 365 (M+1)⁺, retention time: 3.01, ELS-purity: 95%.

### Example 56

4-{7-[2-(4,5-Dihydro-1H-imidazol-2-yl)-1-pyridin-2-yl-ethyl]-2,3-dihydro-benzofuran-5-yl}-benzaldehyde

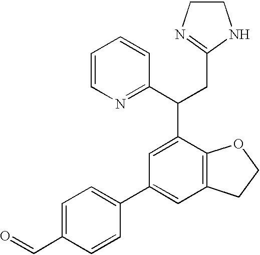

A solution of 2-[1-(5-Bromo-2,3-dihydro-benzofuran-7-yl)-2-(4,5-dihydro-1H-imidazol-2-yl)-ethyl]-pyridine (0.672 mmol, 250 mg), 4-formyl phenyl boronic acid (88-7064) (0.672 mmol, 100,7 mg) and anhydrous K₂CO₃ (186 mg) in toluene (15 mL) and dry EtOH (1.5 mL) was degassed by bubbling N₂ through the solution for 30 min, before adding Pd(PPh₃)₄ (88-1231) (10 mol %, 77 mg). After three cycles of evacuation and re-filling with N₂, the mixture was heated to 80-90° C. for 2 h 30 min. The reaction mixture was cooled to room temperature, quenched with sat. aq. NH₄CI (15 mL), the layers separated and the aqueous one extracted with dichloromethane (3×25 mL). The combined organic fractions were dried over MgSO₄, filtered and evaporated. The crude product was purified by semi preparative HPLC to give pure 4-{7-[2-(4,5-dihydro-1H-imidazol-2-yl)-1-pyridin-2-yl-ethyl]-2,3-dihydrobenzofuran-5-yl}-benzaldehyde as an oil, (73 mg, 27%). LC-MS: retention time: 2.24 min, m/z: 398 (M+1)⁺; ELS purity: 97%. ¹H-NMR (CDCl₃, ppm): 3.03 (dd, 1H); 3.25 (t, 2H); 3.39 (dd, 1H); 3.45 (s, br; 4H); 4.62 (t, 2H); 4.81 (dd, 1H); 7.12 (ddd, 1H); 7.26-7.34 (m, 3H); 7.57 (ddd, 1H); 7.61 (d, 2H); 7.86 (d, 2H); 8.55 (ddd, 1H); 9.99 (s, 1H:CHO). ¹³C-NMR (CDCl₃, ppm): 30.23; 33.40; 44.94; 50.02; 72.01; 122.08; 122.80; 124.04; 125.63; 126.80; 127.45; 128.47; 130.59; 132.78; 134.83; 136.99; 147.65; 149.29; 158.72; 161.92; 167.42; 192.24.

### Example 57

6-Aminohexanoic Acid {7-[2-(4,5-dihydro-1H-imidazol-2-yl)-1-pyridin-2-yl-ethyl]-2,3-dihydrobenzofuran-5-yl}amide

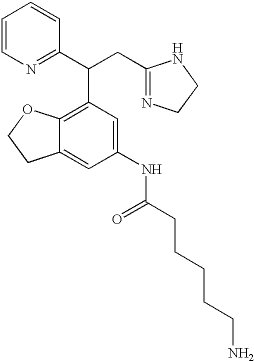

7-[2-(4,5-Dihydro-1H-imidazol-2-yl)-1-pyridin-2-yl-ethyl]-2,3-dihydrobenzofuran-5-ylam ine (0.277 mmol, 85.4 mg) in THF (10 mL) was treated with Boc-epsilon-aminocaproic acid succinimide ester (0.305 mmol, 100 mg) in triethylamine (56 μL) and THF (10 mL). The reaction mixture was stirred overnight at room temperature under nitrogen, the mixture was evaporated to dryness and the residue purified on Flash 40 silica gel column using ethyl acetate:ethanol:triethylamine (4:4:1) as eluent giving 100 mg of an oil which was treated with 0.5 mL trifluoroacetic acid in dichloromethane by stirring at room temperature for 10 min. Subsequently pH was adjusted to 10 by means of NaOH (4 M), the organic fraction was isolated and evaporated to dryness giving 6-aminohexanoic acid {7-[2-(4,5-dihydro-1H-imidazol-2-yl)-1-pyridin-2-yl-ethyl]-2,3-dihydrobenzofuran-5-yl}amide an oil (50 mg). m/z 422 (M+1)⁺, retention time: 0.39 min.

### Example 58

3-[2-(4,5-Dihydro-1H-imidazol-2-yl)-1-pyridin-2-yl-ethyl]benzylamine

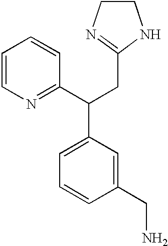

3-[2-(4,5-dihydro-1H-imidazol-2-yl)-1-pyridin-2-yl-ethyl]-benzaldehyde (32 m mol, 9 g), ammonium acetate (200 g, 2.5 mol), and molecular sieves 4 Å (10 g) were mixed in methanol (720 mL) and stirred at room temperature for 18 h. Subsequently Na(OAc)₃BH (16.7 g, 80 mmol) was added in three portions over a period of 1.5 h. After further 1.5 h stirring at room temperature the mixture was evaporated to dryness. Dichloromethane (150 ml) and water (50 ml) were added followed by NaOH (10%) to adjust pH to 11. The organic layer was separated, dried over Na₂SO₄ and concentrated, resulting in 7.8 g of a brown oil, which contained a mixture. The oil was purified by column chromatography (Al₂O₃ nach Brockman) using dichloromethane containing 4% methanol as eluent. The purification resulted in isolation of 3-[2-(4,5-dihydro-1H-imidazol-2-yl)-1-pyridin-2-yl-ethyl]benzylamine (1.4 g), which was isolated as the fumarate salt. ¹H NMR of the free base (CDCl₃) ppm: 8.57(1H,dd); 7.58(1H,dd); 7.3-7.05 (6H, multiplet); 4.57 (1H,dd); 3.79 (2H,s); 3.40 (4H,s); 3.40-3.24 (1H, multiplet); 2.71 (1 H.dd); 3.08-2.71 (2H, broad). LCMS: (M+1)⁺481, retention time: 4.41 m in.

### Example 59

Separation of the Enantiomers of 4-[2-(4,5-dihydro-1H-imidazol-2-yl)-1-(3-methoxy-phenyl)-ethyl]-pyridine

Racemic 4-[2-(4,5-dihydro-1H-imidazol-2-yl)-1-(3-methoxy-phenyl)-ethyl]-pyridine was separated in enantiomers on a Chiralpak® AD column, using 2-propanol:n-heptane:diethylamine, 50:50:0.1 as eluent.

The two enantiomers having the retention time 12.6. min. (93.7% ee) and 16.0 min.(98.5% ee), respectively, were obtained.

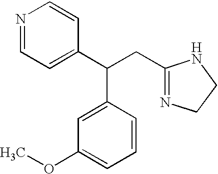

### Example 60

Separation of the Enantiomers of 2-[1-(3-methoxy-phenyl)-2-(1-propyl-4,5-dihydro-1H-imidazol-2-yl)-ethyl]-pyridine

Racemic 2-[1-(3-methoxy-phenyl)-2-(1-propyl-4,5-dihydro-1H-imidazol-2-yl)-ethyl]pyridine was separated in enantiomers on a Chiralpak® AD column, using 2-propanol:n-heptane:diethylamine, 5:95:0.1 as eluent.

The two enantiomers having the retention time 18.8. min. (>95% ee) and 21.0 min. (99.4% ee), respectively, were obtained.

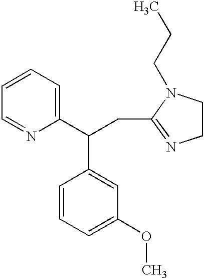

### Example 61

(4-{3-[2-(4,5-Dihydro-1H-imidazol-2-yl)-1-pyridin-2-yl-ethyl]benzyloxy}phenyl)phenylmethanone

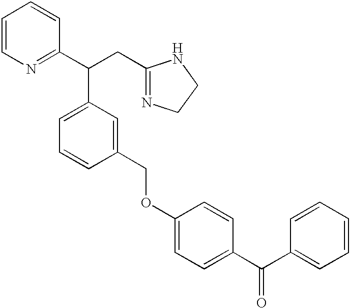

{3-[2-(4,5-Dihydro-1H-imidazol-2-yl)-1-pyridin-2-yl-ethyl]phenyl}methanol (1.78 m mol, 0.5 g) and 4-benzoylphenol (1.78 mmol, 0.35 g) were mixed in dichloromethane (50 mL). PS-triphenylphosphine (load 1.73 mmol/g, 1.03 g) was added and the resulting mixture cooled to 0° C., and diethyl azodicarboxylate (1.78 mmol, 0.342 mL) was added dropwise under nitrogen.

The reaction mixture was heated to room temperature and stirred for 24 h. Subsequently the mixture was filtered, the residue washed 3 times with dichloromethane and the collected dichloromethane phases were extracted with NaOH (1 M) followed by extraction with HCl (1 M).

The resulting organic phase was dried with MgSO₄, filtered and evaporated resulting in an oil which was purified on Flash 40 silica gel column using methanol as eluent. (4-{3-[2-(4,5-Dihydro-1H-imidazol-2-yl)-1-pyridin-2-ylethyl]benzyloxy}phenyl)phenylmethanone.

100 mg of product was isolated as an oil which was further purified with active carbon in dichloromethane solution.

LCMS: m/z (M+1)⁺462, retention time: 3.22 min. ¹³C NMR (CDCl₃) ppm: 195.9, 167.6, 162.6, 162.1, 149.3, 143.8, 138.5, 137.1, 136.9, 132.9, 132.3, 130.6, 130.1, 129.4, 128.6, 128.1, 127.4, 126.4, 124.2, 122.2, 114.8, 70.4,50.8, 49.4, 34.5.

### Example 62

2-Amino-3-(4-benzoyl-phenyl)—N-{3-[2-(4,5-dihydro-1H-imidazol-2-yl)-1-pyridin-2-yl-ethyl]-benzyl}propionamide

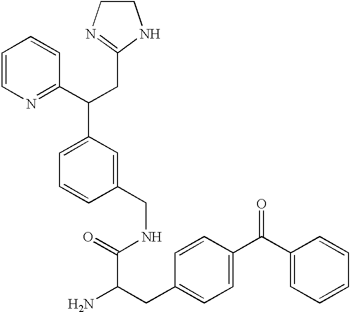

N-Fmoc-4-benzoylphenylalanine (1 mmol, 491 mg) was mixed with HOBT,H₂O (1 mmol, 153 mg) in dichloromethane (20 mL); EDAC,HCl (1.1 mmol, 200 mg) was added and the mixture stirred under nitrogen at room temperature for 2 h. 3-[2-(4,5-Dihydro-1H-imidazol-2-yl)-1-pyridin-2-yl-ethyl]benzylamine (1 mmol, 280 mg) in dichloromethane (4 mL) was added and the mixture stirred overnight at room temperature. The solvent was subsequently evaporated and the residue treated with piperidine:methanol (1:4) (20 mL) by stirring at room temperature for 30 min.

The solvent was evaporated and the residue extracted between NaOH (1 M) and dichloromethane.

The organic layers were collected and evaporated to give an oil. Treatment with methanol caused crystallisation of Fmoc-piperidine, which was isolated by filtration and discarded. The filtrate was purified on a silica gel column yielding 2-amino-3-(4-benzoyl-phenyl)-N-{3-[2-(4,5-dihydro-1H-imidazol-2-yl)-1-pyridin-2-yl-ethyl]benzyl}propionam ide (220 mg) as an oil. LCMS: m/z (M+1)⁺532. Retention time: 1.92 min, purity (ELS) 95%.

### Example 63

4-Benzoyl-N-{3-[2-(4,5-dihydro-1H-imidazol-2-yl)-1-pyridin-2-yl-ethyl]phenyl}benzamide

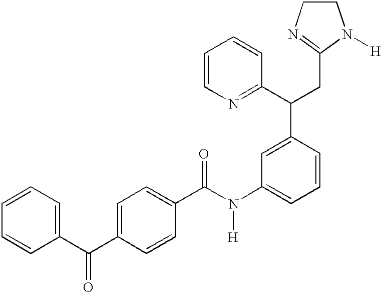

3-[2-(4,5-Dihydro-1H-imidazol-2-yl)-1-pyridin-2-yl-ethyl]phenylamine, fumarate (0.15 mmol, 57 mg), 4-benzoylbenzoic acid succinimide ester (0.15 mmol, 50 mg), and triethylamine (3 mL) were mixed in THF (20 mL). The mixture was stirred for 6 days. Subsequently the mixture was filtered and the filtrate evaporated to dryness. This was extracted between NaOH (1 M) and dichloromethane and the organic layer was isolated, dried over MgSO₄ and evaporated to dryness to give an oil. This was further purified on a silica gel column using dichloromethane:MeOH (9:1) as eluent. 4-Benzoyl-N-{3-[2-(4,5-dihydro-1H-imidazol-2-yl)-1-pyridin-2-yl-ethyl]-phenyl}benzamide (30 mg oil) was isolated. ¹³C NMR (CDCl₃) PPM: 196.3, 168.1,162.7, 160.9, 149.2, 146.9,144.6, 140.3, 140.0, 137.3, 136.6, 133.3, 130.5, 130.4, 129.7, 128.9, 127.6, 123.7, 121.7, 118.7, 115.1, 113.9, 53.4, 50.8, 49.5, 36.5

LCMS: m/z (M+1)⁺475, retention time: 2.38 min.

### Example 64

5-(2-Oxo-hexahydro-thieno[3,4-d]imidazol-6-yl)-pentanoic Acid (2-(4-benzoyl-phenyl)-1-{3-[2-(4,5-dihydro-1H-imidazol-2-yl)-1-pyridin-2-yl-ethyl]benzylcarbamoyl}ethyl)amide

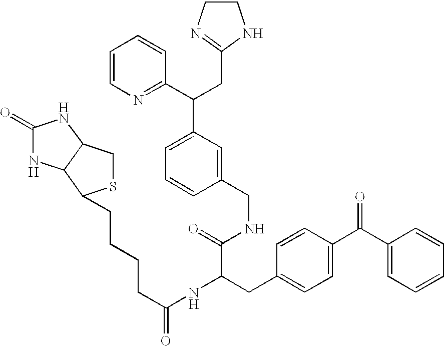

2-Amino-3-(4-benzoyl-phenyl)-N-{3-[2-(4,5-dihydro-1H-imidazol-2-yl)-1-pyrid in-2-yl-ethyl]-benzyl}propionamide (0.129 mmol, 68.5 mg) was dissolved in DMF (1 mL) and mixed with D-Biotinyl-Osu (0.129 mmol, 44 mg) dissolved in DMF (1 mL). Saline phosphate buffer (pH 7.4) (0.5 mL) was added and the mixture was stirred overnight. The solvent was evaporated to give an oil (100 mg). 50 mg of this oil was purified on HPLC resulting in 5-(2-oxohexahydrothieno[3,4-d]imidazol-6-yl)pentanoic acid (2-(4-benzoylphenyl)-1-{3-[2-(4,5-d ihydro-1H-imidazol-2-yl)-1-pyrid in-2-yl-ethyl]-benzylcarbamoyl}ethyl)am ide (8.2 mg).

LCMS: m/z (M+1)⁺759, retention time: 2.42 min.

### Example 65

4-[2-(4,5-Dihydro-1H-imidazol-2-yl)-1-(3-methoxy-phenyl)ethyl]pyridine

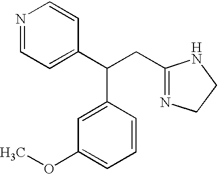

2.5 g of Z,E-2-[2-(3-methoxyphenyl)-2-pyridin-4-yl-vinyl]-4,5-dihydroimidazole-1-carboxylic acid tert-butyl ester compound (6.6 mmol) was hydrogenated by shaking overnight in ethanol (100 mL) using Pd/C (10%, 250 mg) as catalyst and a hydrogen pressure of 60 psi resulting in 2-[2-(3-methoxy-phenyl)-2-pyridin-4-yl-ethyl]-4,5-dihydroimidazole-1-carboxylic acid tertbutyl ester as a pale yellow oil (2.36 g, 94%). LCMS : m/z 382 (M+1)+; retention time: 1.97 min. ¹H NMR (CDCl₃, ppm):1.50 (s,9H); 3.48(t,2H); 3.63(s,broad,4H); 3.76(s,3H); 4.61(t,1H); 6.71-6.86(m,3H); 7-16-7.24 (m,3H); 8.4-8.49(m,2H). ¹³C NMR (CDCl₃, ppm): 27.9; 35.2; 46,5; 47.1; 51.8; 54.9; 81.5; 111.5; 113.9; 120.0; 123.0; 129.3; 143.9; 149.5; 150.7; 152.7; 158.6; 159.4. 2.27 g of the product from the reduction was de-BOC'ed in HCl (26 mL, 3 N) and ethyl acetate (26 mL) by stirring 3 days at room temperature. pH was adjusted to 10 (NaOH, 10 M) the organic phase was separated and dried (MgSO₄) and evaporated to dryness to give 4-[2-(4,5-dihydro-1H-imidazol-2-yl)-1-(3-methoxy-phenyl)ethyl]pyridine as a yellow oil (1.17 g, 71%). LCMS: retention time: 0.62 min; m/z 282 (M+1)⁺. ¹H NMR (CDCl₃) ppm: 2.79(m,2H); 3.30(s,4H); 3.56(s,3H); 4.37(t,1H); 5.56(s,broad,1H); 6.58-6.67(m,3H); 7.01-7.09(m,3H) 8.30(d,2H).

¹³C NMR (CDCl₃) ppm: 34.9; 47.8; 49.8; 55.3; 112.1; 114.2; 120.1; 123.3; 130.0; 144.1; 150.0; 152.9; 160.0; 166.0.

### Pharmacological Methods

### Effect of the Compounds of the Invention on Isolated α-Cells and β-Cells

The effect of the compounds of the invention on isolated α-cells and β-cells can be investigated as exemplified below with the title compound of Example 2 (compound (Ex 2).

Preparation of Islets and Single β-Cells

Pancreatic islets were isolated from fed NMRI mice (15-23 g). Briefly, the mice were stunned by a blow against the head and killed by cervical dislocation. The pancreas was quickly removed and pancreatic islets were isolated by collagenase (Sigma) digestion (Gromada et al. J. Physiol., Vol. 518 (3) pp. 745-759, 1999). For insulin release experiments, the islets were kept in RPMI-1640 tissue culture medium overnight before use. Alternatively, the islets were dispersed into single cells by shaking in a Ca²⁺-free solution and the resulting cell suspension was plated on Nunc petri dishes and maintained for up to 3 days in RPMI-1640 medium supplemented with 10% heat-inactivated fetal calf serum, 100 i.u./ml penicillin and 100 μg/mI streptomycin.

Preparation of Single α-Cells

Male Lewis rats (250-300 g; Møllegaard, Lille Skensved, Denmark) were anaesthetised by pentobarbital (100 mg/kg i.p.). After removal of the pancreas, islets were isolated by collagenase digestion and dispersed into single cells using dispase (Sigma). The α-cells were then separated by fluorescence-activated cell sorting as described elsewhere (Josefsen et al. J. Endocrinol., Vol.149, pp.145-154, 1996). Based on the hormone contents and their glucose sensitivities, we estimate that the preparation contains >80% α-cells and <3% β-cells. The cell suspension was plated on 35-mm diameter petri dishes and incubated in a humidified atmosphere for up to five days in RPMI 1640 tissue-culture medium (Gibco BRL, Life Technologies Ltd, Paisley, UK) supplemented with 10% (v/v) heat-inactivated fetal calf serum, 100 lU/ml penicillin and 100 μg/ml streptomycin.

Electrophysiology

Patch pipettes were drawn from borosilicate glass capillaries. The tips of the pipettes were coated with SYLGARD® (Dow Corning, USA) and fire-polished before use. The pipette resistance (when filled with the pipette-filling solutions) was 2-4 MΩ. All currents have been filtered at 1 kHz using the internal filters of the amplifiers and acquired at a rate of 5 kHz. The zero-current potential was adjusted before establishment of the seal with the pipette in the bath. The whole-cell K_{ATP} conductance was estimated by applying 10 mV hyper- and depolarizing voltage pulses (duration: 200 ms; pulse interval: 2 s) from a holding potential of -70 mV using the standard whole-cell configuration of the patch-clamp technique. The currents were recorded using an Axopatch 200B patch clamp amplifier (Axon Instruments, Foster City, Calif., USA), digitized and stored in a computer using the Digidata AD-converter and the software pClamp (version 6.0; Axon Instruments). Alternatively, K_{ATP}-currents were recorded using an EPC-9 patch clamp amplifier and the Pulse software (v. 8.01; HEKA Elektronik, Lamprecht/Pfalz, Germany). Exocytosis was monitored as increases in cell membrane capacitance (Neher & Marty Proc. Natl. Acad, Sci. USA, Vol. 79, pp. 6712-6716,1982) using the standard whole-cell configuration of the patch-clamp technique and an EPC-9 patch-clamp amplifier and the Pulse software (v. 8.01; HEKA Elektronik). The interval between two successive points was 0.4 s. The measurements of cell capacitance were initiated <5 seconds after the whole-cell configuration was established. Exocytosis was elicited by infusion of Ca²⁺-EGTA buffers through the recording electrode. The extracellular medium consisted of 138 mM NaCl, 5.6 mM KCl, 2.6 mM CaCl₂, 1.2 mM MgCl₂, 5 mM HEPES (N-[2-hydroxyethyl]piperazine-N′-[2-ethanesulfonic acid) and 5 mM D-glucose (pH 7.4 with NaOH). The volume of the recording chamber was 0.4 ml and the solution entering the bath (1.5-2 ml/min) was maintained at +33° C. for measurements of exocytosis. For recordings of whole-cell K_{ATP}-channel activity, the pipette solution contained 125 mM KCl, 30 mM KOH, 10 mM EGTA, 1 mM MgCl₂, 5 mM HEPES, 0.3 mM Mg-ATP and 0.3 mM K-ADP (pH 7.15 with KOH). The electrode solution for measurements of exocytosis consisted of 125 mM potassium glutamate, 10 mM KCl, 10 mM NaCl, 1 mM MgCl₂, 5 mM HEPES, 3 mM Mg-ATP,10 mM EGTA and 5 or 9 mM CaCl₂. The free Ca²⁺ concentration of the resulting buffer were 0.22 and 2 μM using the binding constants of Martell & Smith (Martell, A. E. & Smith, R. M.: Critical Stability Constants, vol 1, Amino Acids, and vol 2, Amines., 1971. Plenum Press, New York).

Insulin Release

Intact pancreatic islets were isolated from fed NMRI mice (15-18 g) as previously described (Fuhlendorff et al., Diabetes, Vol. 47 (3) pp. 345-351, 1998). Insulin release was measured from groups of 10 size-matched islets, cultured overnight in RPMI-1640 tissue culture medium containing 1% GlutaMAX™ (Gibco BRL),1% penicillin/streptomycin, 7.5% NaHCO₃ and 10% new-born calf serum. The islets were washed for 20 minutes in Krebs Ringer Buffer consisting of 115 mM NaCl, 4.7 mM KCl, 2.6 mM CaCl₂, 1.2 mM KH₂PO₄, 1.2 mM MgSO₄, 20 mM HEPES, 2 mM glutamine, 5 mM NaHCO₃, 0.2% human serum albumin and 1% penicillin/streptomycin) and 2.5 mM, 5 mM or 10 mM D-glucose. Afterwashing, the islet were incubated for one hour at +37° C. in Krebs Ringer Buffer containing the test compound at 100 μM and glucose at 2.5 mM, 5 mM or 10 mM. After the one-hour incubation, the medium was aspirated and kept at −20° C. until assayed for insulin using the following ELISA technique. Immuno 96 well paltes (MaxiSorPTM, NUNC, Denmark) were coated over night at 4° C. with a rabbit-anti-guinea pig-lgG (Dako) antibody diluted 1:1000 in 0.1 M NaHCO₃ (pH 9.8). After washing 4 times in 0.15 M NaCl and 0.005% Tween-20 the plates were incubated with guinea pig-anti-insulin diluted in phosphate buffered saline (incubation buffer; pH 7.4) containing 0.1% Tween-20 and 0.5% human serum albumin over night. After washing the sambles were incubated for 1.5 h with relevant samples and peroxidase-insulin (Sigma) diluted 1:10000 in PBS-DIL. The samples we-were then washed and developed with TMBPlus-substrated as described by the manufacture (KEM EN TEC, Denmark). The amount of insulin was quantified from a standard curve after reading the samples at 450 nm.

Data Analysis

The exocytotic rate, ΔCm/Δt, is presented as the increase in cell capacitance occurring during the first 60 seconds following establishment of the whole-cell configuration excluding any rapid changes occurring during the initial 5-10 seconds required for equilibration of the pipette solution with cytosol. Results are presented as mean values ±S.E.M. for the indicated number of experiments/cells. Statistical significance was evaluated using Student's t-test.

Results

Compound (Ex 2) Stimulates Ca²⁺-Dependent Exocytosis in Mouse Pancreatic β-Cells

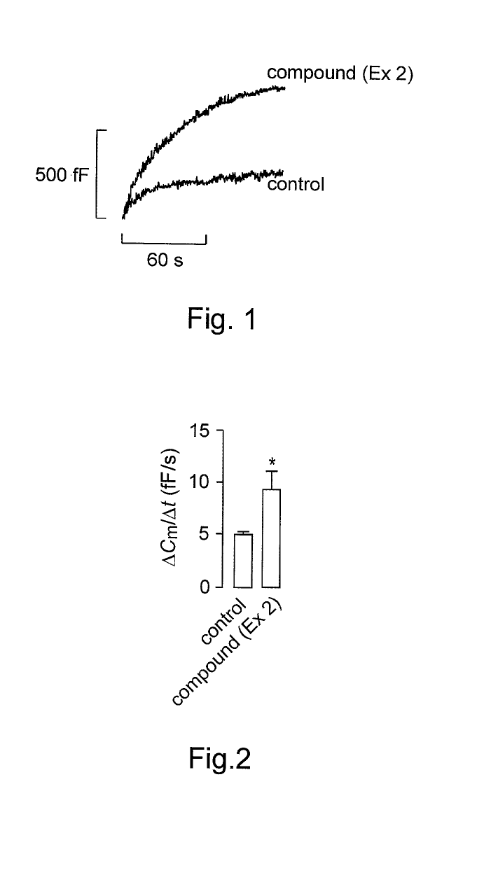

The effects of compound (Ex 2) on Ca²⁺-evoked exocytosis in mouse pancreatic β-cells were investigated in standard whole-cell experiments in which secretion was evoked by intracellular dialysis with a Ca²⁺-EGTA buffer with a free Ca²⁺ concentration of 0.2 μM. Following establishment of the whole-cell configuration, exocytosis was observed as a gradual capacitance increase (FIG. 1; control). In general, the cell capacitance reached a new steady-state level within 3-4 minutes. Inclusion of 100 μM compound (Ex 2) in the pipette solution produced a strong stimulation of exocytosis (FIG. 1; compound (Ex 2)). On average (FIG. 2), the rate of exocytosis measured over the first 60 s after establishment of the whole-cell patch configuration increased from 5.0±0.3 fF/s (n=1 3) under control conditions to 9.4±1.7 fF/s (P<0.025; n=7) in the presence of compound (Ex 2).

Compound (Ex 2) does not Affect ATP-Sensitive K⁺-Channel Activity

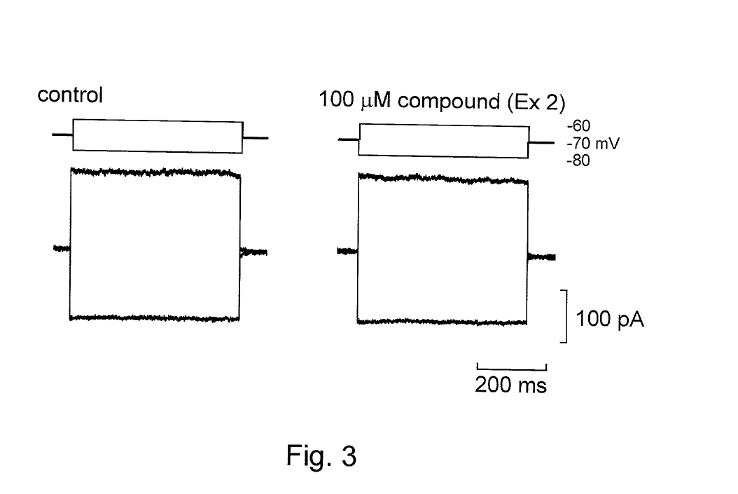

The stimulatory action of compound (Ex 2) on exocytosis was not associated with closure of the plasma membrane K_{ATP}-channels. Changes in whole-cell K_{ATP} currents in single mouse pancreatic p-cells were measured in response to 10 mV depolarising and repolarising voltage pulses from a holding potential of −70 mV. The cells were dialysed with 0.3 mM ATP and 0.3 mM ADP to activate the K_{ATP}-channels. FIG. 3 depicts representative K_{ATP} currents measured before (control) and after the addition of either 100 μM compound (Ex 2). It is clear that compound (Ex 2) did not affect channel activity and in a series of six experiments, channel activity amounted to 90±4% of the prestimulatory level.

Effects of Compound (Ex 2) on Insulin Secretion from Intact Mouse Islets

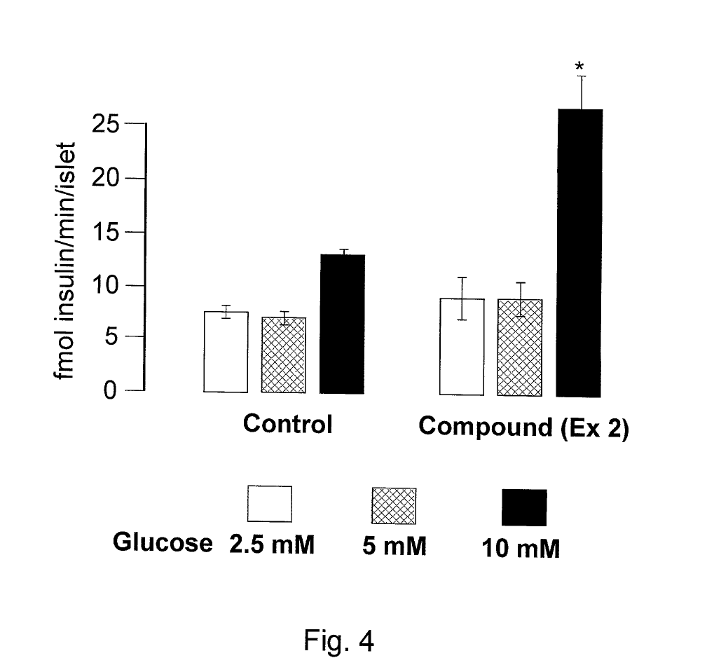

FIG. 4 shows measurements of insulin release from groups of 10 size-matched islets in the presence of 2.5 mM, 5 mM and 10 mM glucose. Elevating the external glucose concentration from 2.5 mM to 5 mM was not associated with an increase in insulin release whereas a 90% stimulation was observed following application of 10 mM glucose (P<0.05; n=8). The histogram in FIG. 4 clearly demonstrates that 100 μM compound (Ex 2) does not affect insulin release at 2.5 or 5 mM glucose but stimulates insulin secretion at 10 mM glucose by 220% (P<0.01; n=8).

Compound (Ex 2) Inhibits Exocytosis in Rat Pancreatic α-Cells

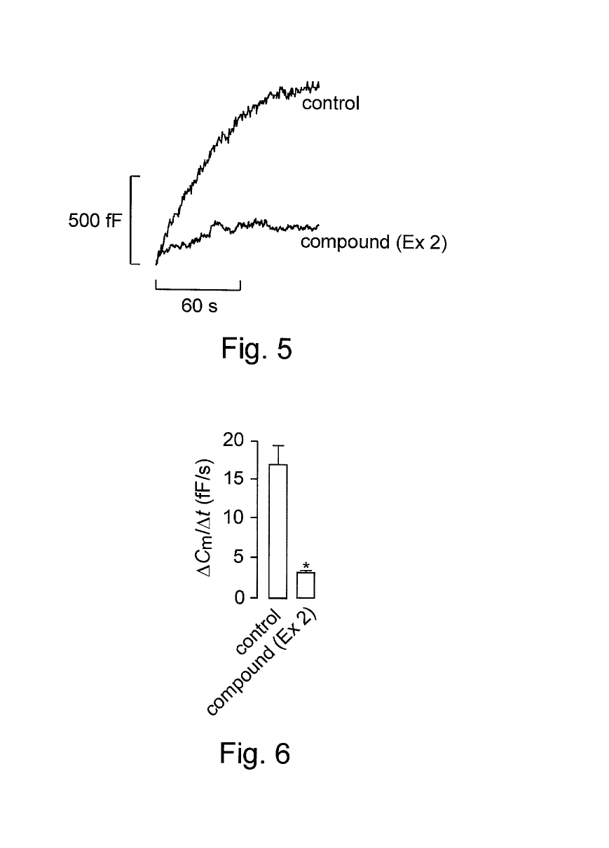

FIG. 5 shows that compound (Ex 2) inhibits Ca²⁺-evoked exocytosis in rat pancreatic α-cells in standard whole-cell experiments in which secretion was evoked by intracellular dialysis with a Ca²⁺-EGTA buffer with a maximal free Ca²⁺ concentration of 2 μM. Following establishment of the whole-cell configuration, exocytosis was observed as a strong capacitance increase (control). Inclusion of 100 μM compound (Ex 2) in the pipette solution inhibited exocytosis (compound (Ex 2)). On average (FIG. 6), the rate of exocytosis measured over the first 60 s after establishment of the whole-cell patch configuration decreased from 16.8±2.5 fF/s (n=5) under control conditions to 4.6±0.7 fF/s (P<0.001; n=5) in the presence of compound (Ex 2).

In summary, these data suggest that compound (Ex 2) stimulates exocytosis in pancreatic p-cells without affecting the activity of the ATP-sensitive K⁺ channels leading to a glucose-dependent stimulation of insulin release. Furthermore, compound (Ex 2) inhibits exocytosis in pancreatic a-cells.

Effect of the Compounds of the Invention on Insulin Release from Isolated Perfused Rat Pancreas

The effect of the compounds of the invention on the insulin release from isolated perfused rat pancreas can be investigated as exemplified below with the title compound of Example 2 (compound (Ex 2). In the example described, the investigation is carried out at 2.8 mM and 8.3 mM glucose.

Materials and Methods:

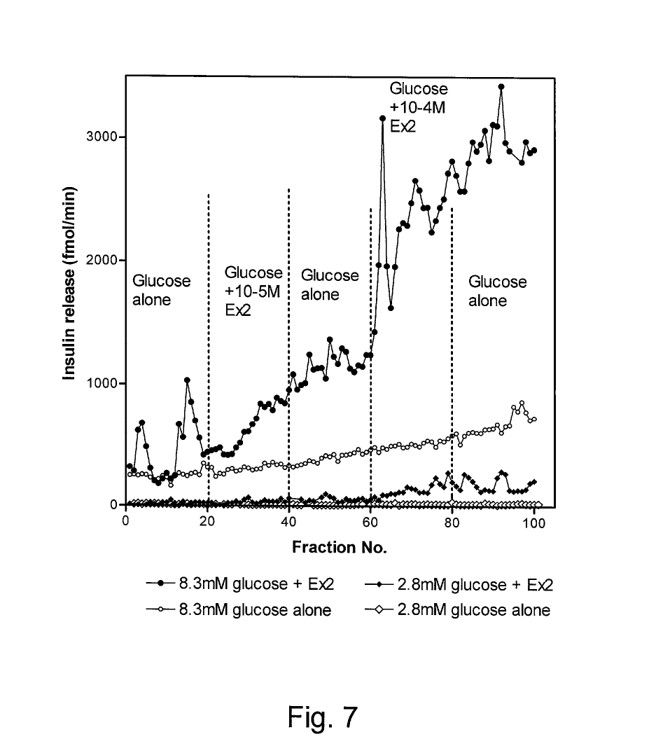

Male Sprague Dawley rats (body weight around 300 g) were anaesthetised with a mixture of Hypnorm® (Janssen Pharma) and Dormicum® (Roche) and the pancreas was cannulated and isolated a described by Sturis et al., Am. J. Physiol. 269, E786-E792, 1995. The organ was placed at 37° C. in a humidified perfusion chamber and perfused with a 5% 02/95% CO₂ gassed bicarbonated Krebs Ringer, 4% Dextran buffer from a pump delivering 71% of the total volume and from a pump delivering 29% volume of the same buffer with added BSA (0.2% final concentration) and glucose (2.8 mM or 8.3 mM final concentration). The 29% pump also delivered compund (Ex 2) at 10⁻⁵ M in the interval t=20-40 min and at 10⁻⁴ M in the interval t=60-80 min. The pancreata were acclimatised for 20 min before start of sampling at 1 fraction of ca 1900 μl/min for 100 min. Aliquots of 200 μl were frozen away at −20° C. for subsequent insulin ELISA using guinea pig anti-insulin antibodies. The results are shown in FIG. 7 and are expressed as fmol insulin released per pancreas per min. Each plot represents one pancreas, except 8.3 mM glucose alone mean of n=2. The graph shows that initial insulin secretion was higher in 8.3 mM glucose than in 2.8 mM glucose (ca 300 vs 20 fmol/min). In the pancreas infused with 8.3 mM glucose and compound (Ex 2) there was an initial noise in the interval t=0-20 min which was due to a mechanical problem with the tubing. From 2040 min (10⁻⁵ M compound (Ex 2)) there was a small increase in insulin secretion that did not revert when the compound was removed at t=40 min. When 10⁻⁴ M compound (Ex 2) was added at t=60 min there was a classic first-phase insulin release peaking at 3000 fmol/min, followed by second-phase release. However, again the secretion did not revert when compound (Ex 2) was removed at t=80 min. We have evidence that non-reversal may be due to the low BSA concentration (0.2%) in the perfusate. Insulin from pancreas perfused with 8.3 mM glucose alone showed a slow increase from 300 up to ca 700 fmol/min by t=100 min.

In the pancreas perfused with compound (Ex 2) at 2.8 mM glucose, the insulin secretion was only about 10% of that seen at 8.3 mM glucose (max. 250 fmol/min) with 10⁻⁴ M compound (Ex 2) while the control pancreas secretion was flat at ca 20 fmol/min. The data suggest that insulin secretion by compound (Ex 2) is glucose dependent.

1. A compound of formula (I) 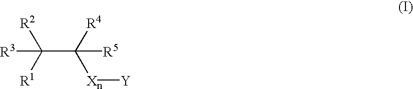
wherein Y is selected from the following ring systems: 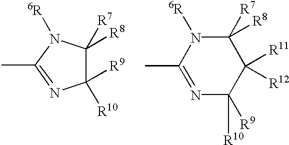
R¹ and R² are independently phenyl, naphthyl, thienyl, thiazolyl, imidazolyl, pyrazolyl, triazolyl, pyridyl, pyrimidyl, pyridazinyl, pyrazinyl, quinolyl, 3,4-dihydroquinolyl, 5,6,7,8-tetrahydroquinolyl, isoquinolyl, 3,4-dihydroisoquinolyl, 5,6,7,8-tetrahydroisoquinolyl, indolyl, benzo[b]thienyl, benzimidazolyl, 5,6,7,8-tetrahydroimidazo[1,5-a]pyridyl or benzthiazolyl each of which is optionally substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R ¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;
R³ and R⁵ are independently hydrogen, C₁₋₆alkyl or halogen or when taken together R³ and R⁵ form an additional bond between the carbon atoms to which they are attached;
R⁴ is hydrogen, C₁₋₆alkyl or halogen;
R⁶ is hydrogen, C₁₋₆alkyl, arylC₁₋₆alkyl, C₁₋₆alkyl-C(O)— or arylC₁₋₆alkyl-C(O)— wherein the alkyl groups are optionally substituted with halogen, methoxy or ethoxy and the aryl groups are optionally substituted with halogen, methyl, ethyl, methoxy or ethoxy;
R⁷, R⁸, R⁹, R¹⁰, R¹¹, and R¹² are independently hydrogen, C₁₋₆alkyl, aryl or arylC₁₋₆alkyl;
wherein the alkyl groups are optionally substituted with halogen, methoxy or ethoxy and the aryl groups are optionally substituted with halogen, methyl, ethyl, methoxy or ethoxy;
X is —CR¹⁸R¹⁹— wherein R¹⁸ and R¹⁹ are independently hydrogen or C₁₋₆alkyl;
n is 0, 1 or 2; and any stereoisomers and geometrical isomers and salts thereof with the proviso that the compound of formula (I) is not one of the following compounds:
2-(2,2-diphenylethyl)-4,5-dihydro-1H-imidazole;
2-[2-phenyl-2-(2-pyridyl)]ethyl-2-imidazoline;
2-[2-(4-chlorophenyl)-2-(2-pyridyl)]ethyl-2-imidazoline;
2-[2-(indol-3-yl)-2-phenyl]ethyl-2-imidazoline;
2-[2-(2-methylindol-3-yl)-2-phenyl]ethyl-2-imidazoline;
2-[2-(indol-3-yl)-2-(3-methoyxphenyl)]ethyl-2-imidazoline;
2-[2-(2-methylindol -3-yl)-2-(3-methoxyphenyl)]ethyl-2-imidazoline;
2-[2-(5-chloro-2-methylindol-3-yl)-2-(3-methoxyphenyl)]ethyl-2-imidazoline;
2-[2-(4-chlorophenyl)-2-phenylethyl]-4,5-dihydro-1H-imidazole;
2-[2-(4-chlorophenyl)-2-phenylethyl ]-4,5-dihydro-1H-imidazole;
2-[2-(3-tolyl)-2-phenylethyl]-4,5-dihydro-1H-imidazole;
2-[2,2-bis(4-hydroxyphenyl)ethyl]-4,5-dihydro-1H-imidazole;
2-(2,2-di phenylethyl)-4,5-dihydro-1-methyl-1H-imidazole;
2-(2, 2-diphenylethyl)-4,5-dihydro-4-methyl-1H-imidazole;
4-[2-(4,5-dihydro-1H-imidazol-2-yl)-1-phenylethyl]pyridine;
2-(2,2-diphenylethyl)-4,5-dihydro-1H-imidazole-1-carboxaldehyde;
1-acetyl-2-(2,2-diphenylethyl)-4,5-dihydro-1H-imidazole;
2-(2,2-diphenylethyl)-4,5-dihydro-1H-imidazole-1-carboxylic acid ethyl ester;
2-(2,2-diphenylethyl)-4,5-dihydro-1-hydroxymethyl-1H-imidazole;
2-(2,2-diphenylethyl)-1,4,5,6-tetrahydropyrimidine; and
2-(2-(4,5-dihydro-1H-imidazol-2-yl)-1-thiophene-2-yl-ethyl)pyridine.

2. A compound of formula (I) 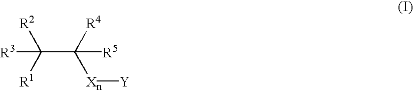
wherein Y is selected from the following ring systems: 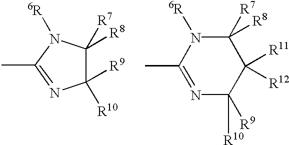
R¹ and R² are independently naphthyl, 3-thienyl, thiazolyl, imidazolyl, pyrazolyl, triazolyl, 3-pyridyl, pyrimidyl, pyridazinyl, pyrazinyl, quinolyl, 3,4-dihydroquinolyl, 5,6,7,8-tetrahydroquinolyl, isoquinolyl, 3,4-dihydroisoquinolyl, 5,6,7,8-tetrahydroisoquinolyl, 4-indolyl, 7-indolyl, benzo[b]thienyl, benzimidazolyl, 5,6,7,8-tetrahydroimidazo[1,5-a]pyridyl or benzthiazolyl each of which is optionally substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;
with the further option that R¹ or R² or both R¹ and R² are selected from the group consisting of 2-thienyl, 2-pyridyl and 4-pyridyl substituted with C₁₋₆alkyl, hydroxy, C₁₋₆alkoxy, cyano, trifluoromethoxy, halogen, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;
with the still further option that R¹ or R² or both R¹ and R² are selected from the group consisting of phenyl substituted with C₂₋₆alkyl, hydroxy, C₁₋₆alkoxy, cyano, trifluoromethoxy, fluorine, bromine, iodine, trifluoromethyl, nitro, COOR¹³, —NR¹³R¹⁴ wherein R¹³ and R¹⁴ independently are hydrogen, C₁₋₆alkyl, aralkyl, —C(O)—R¹⁵ wherein R¹⁵ is C₁₋₆alkyl or arylC₁₋₆alkyl, or carboxamide of the formula —C(O)—NR¹⁶R¹⁷ wherein R¹⁶ and R¹⁷ independently are hydrogen, C₁₋₆alkyl, arylC₁₋₆alkyl or the R¹³R¹⁴ and R¹⁶R¹⁷ groups may independently be taken together with the nitrogen to which they are attached forming a saturated, partially saturated or aromatic monocyclic or bicyclic ring system containing 3 to 14 carbon atoms and 0 to 3 additional heteroatoms selected from nitrogen, oxygen or sulfur, the ring system optionally being substituted with at least one C₁₋₆alkyl, aryl, aralkyl or oxo substituent;
R³ and R⁵ are independently hydrogen, C₁₋₆alkyl or halogen or when taken together R³ and R⁵ form an additional bond between the carbon atoms to which they are attached;
R⁴is hydrogen, C₁₋₆alkyl or halogen;
R⁶ is hydrogen, C₁₋₆alkyl, arylC₁₋₆alkyl, C₁₋₆alkyl-C(O)— or arylC₁₋₆alkyl-C(O)— wherein the alkyl groups are optionally substituted with halogen, methoxy or ethoxy and the aryl groups are optionally substituted with halogen, methyl, ethyl, methoxy or ethoxy;
R⁷, R⁸, R⁹, R¹⁰, R¹¹, and R¹² are independently hydrogen, C₁₋₆alkyl, aryl or arylC₁₋₆alkyl;
wherein the alkyl groups are optionally substituted with halogen, methoxy or ethoxy and the aryl groups are optionally substituted with halogen, methyl, ethyl, methoxy or ethoxy;
X is —CR¹⁸R¹⁹— wherein R¹⁸ and R¹⁹ are independently hydrogen or C₁₋₆alkyl;
n is 0, 1 or 2; and any stereoisomers and geometrical isomers and salts thereof.

3. A compound according to claim 1 wherein R¹ and R² are identical groups.

4. A compound according to claim 1 wherein the groups R¹ and R² are different from each other.

5. A compound according to claim 1 wherein one of R¹ and R² is an optionally substituted carbocyclic aryl group and the other one is an optionally substituted heterocyclic aryl group.

6. A compound according to claim 1 wherein R³ and R⁵ are hydrogen.

7. A compound according to claim 1 wherein R³, R⁴ and R⁵ are hydrogen.

8. A compound according to claim 1 wherein R³ and R⁵ together form an additional bond between the carbon atoms to which they are attached.

9. A compound according to claim 1 wherein n is 0.

10. A compound according to claim 1 wherein n is 1.

11. A compound according to claim 1 wherein n is 2.

12. A compound according to claim 1 wherein Y is 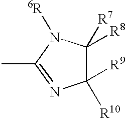
and R⁶, R⁷, R⁸, R⁹ and R¹⁰ are as defined in claim 1.

13. A compound according to claim 12 wherein R⁶, R⁷, R⁸, R⁹ and R¹⁰ are hydrogen.

14. A compound according to claim 12 wherein one of R⁶, R⁷, R⁸, R⁹ and R¹⁰ is C₁₋₆alkyl and the remaining groups are hydrogen.

15. A compound according to claim 12 wherein one of R⁶, R⁷, R⁸, R⁹ and R¹⁰ is methyl and the remaining groups are hydrogen.

16. A compound according to claim 12 wherein one of R⁶, R⁷, R⁸, R⁹ and R¹⁰ is arylC₁₋₆alkyl and the remaining groups are hydrogen.

17. A compound according to claim 12 wherein one of R⁶, R⁷, R⁸, R⁹ and R¹⁰ is benzyl and the remaining groups are hydrogen.

18. A compound according to claim 1 wherein R′ is a heteroaryl group, R² is 3-methoxyphenyl and n is 0.

19. A compound according to claim 18 wherein the heteroaryl group is pyridyl.

20. A compound according to claim 1 wherein R¹ is optionally substituted 2-thienyl and R² is a substituted carbocyclic or heterocyclic group.

21. A compound according to claim 1 wherein R¹ is optionally substituted 3-thienyl and R² is a substituted carbocyclic or heterocyclic group.

22. A compound according to claim 1 wherein R² is 2-chlorophenyl and R² is an optionally substituted carbocyclic or heterocyclic group.

23. A compound according to claim 1 wherein R¹ is 3-chlorophenyl and R² is an optionally substituted carbocyclic or heterocyclic group.

24. A compound according to claim 1 wherein R¹ is 4-chlorophenyl and R² is a substituted carbocyclic or heterocyclic group.

25. A compound according to claim 1 wherein Y is 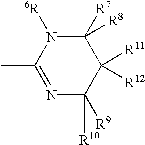
and R⁶, R⁷, R⁸, R⁹, R¹⁰, R¹¹ and R¹² are as defined in claim 1.

26. A compound according to claim 25 wherein R⁶, R⁷, R⁸, R⁹, R¹⁰, R¹¹ and R¹² are hydrogen.

27. A compound according to claim 25 wherein one of R⁶, R⁷, R⁸, R⁹, R¹⁰, R¹¹ and R¹² is C₁₋₆alkyl and the remaining groups are hydrogen.

28. A compound according to claim 25 wherein one of R⁶, R⁷, R⁸, R⁹, R¹⁰, R¹¹ and R¹² is methyl and the remaining groups are hydrogen.

29. A compound according to claim 25 wherein one of R⁶, R⁷, R⁸, R⁹, R¹⁰, R¹¹ and R¹² is arylC₁₋₆alkyl and the remaining groups are hydrogen.

30. A compound according to claim 25 wherein one of R⁶, R⁷, R⁸, R⁹, R¹⁰, R¹¹ and R¹² is benzyl and the remaining groups are hydrogen.

31. A compound selected from the group consisting of:
2-(2-(4,5-Dihydro-1H-imidazol-2-yl)-1-(2-methoxyphenyl)ethyl)pyridine;
2-(2-(4,5-Dihydro-1H-imidazol-2-yl)-1-(2-pyridyl)ethyl)pyridine;
3-(2-(4,5-Dihydro-1H-imidazol-2-yl)-1-phenylethyl)-5,6,7,8-tetrahydroimidazo[,5-a]pyridine;
2-(2-(4,5-Dihydro-1H-imidazol-2-yl)-1-naphthalen-1-ylethyl)pyridine;
1-(2-(4,5-Dihydro-1H-imidazol-2-yl)-1-pyridin-2-ylethyl)isoquinoline;
2-(2-(4,5-Dihydro-1H-imidazol-2-yl)-1-pyridin-2-ylethyl)qui noline;
2-(2-(4,5-Dihydro-1H-imidazol-2-yl)-1-phenylethyl)-1-methyl-1H-imidazole;
2-(2-(4,5-Dihydro-1H-imidazol-2-yl)-1-(2-methoxymethylphenyl)ethyl)pyridine;
2-(2-(4,5-Dihydro-1H-imidazol-2-yl)-1-(2-ethy lphenyl)ethyl)pyridine;
2-(2-(4,5-Dihydro-1H-imidazol-2-yl)-1-(3-methoxyphenyl)ethyl)pyridine;
5-Bromo-2-(2-(4,5-dihydro-1H-imidazol-2-yl)-1-phenylethyl)-1-methyl-1H-imidazole;
2-(2-(4,5-Dihydro-1H-imidazol-2-yl)-1-(2-methoxy-5-methylphenyl)ethyl)pyridine;
2-(2-(4,5-Dihydro-1H-imidazol-2-yl)-1-thiazol-2-ylethyl)pyridine;
2-(2-(4,5-Dihydro-1H-imidazol-2-yl)-1-(2,5-dimethoxyphenyl)ethyl)pyridine;
2-(2-(4,5-Dihydro-1H-imidazol-2-yl)-1-(3,5-dimethoxyphenyl)ethyl)pyridine;
3-(2-(4,5-Dihydro-1H-imidazol-2-yl)-1-thiophen-2-ylethyl)pyridine;
4-(2-(4,5-Dihydro-1H-imidazol-2-yl)-1-thiophen-3-ylethyl)pyridine;
4-(2-(4,5-Dihydro-1H-imidazol-2-yl)-1-thiophen-2-ylethyl)pyridine;
2-(2-(4,5-Dihydro-1H-imidazol-2-yl)-1-(3,4-dimethoxyphenyl)ethyl)pyridine;
2-(2-(4,5-Dihydro-1H-imidazol-2-yl)-1-(3-ethox yphenyl)ethyl)pyridine;
3-Chloro-2-(2-(4,5-dihydro-1H-imidazol-2-yl)-1-(4-methoxyphenyl)ethyl)-5-trifluoromethylpyridine;
1-{2-[2-(3-M ethoxyphenyl)-2-pyridine-2-ylethyl]-4,5-dihydroimidazol-1-yl}ethanone;
{2-[2-(3-Methoxyphenyl)-2-pyridine-2-ylethyl]-4,5-dihydroimidazol-1-yl}phenylmethanone;
2-[1-Benzo[1,3]dioxol-5-yl-2-(4,5-dihydro-1H-imidazol-2-yl)ethyl]pyridine;
2-[2-(4,5-Dihydro-1H-imidazol-2-yl)-1-(3-methoxyphenyl)ethyl]quinoline;
1-[2-(4,5-Dihydro-1H-imidazol-2-yl)-1-(3-methoxyphenyl)ethyl]isoquinoline;
2-[1-(3-Chlorophenyl)-2-(4,5-dihydro-1H-imidazol-2-yl)ethyl]pyridine;
1-[2-(4,5-Dihydro-1H-imidazol-2-yl)-1-(3-methoxyphenyl)vinyl]isoquinoline;
2-[2-(4,5-Dihydro-1H-imidazol-2-yl)-1-(3-methoxyphenyl)vinyl]quinoline;
2-[1-Benzo[1,3]dioxol-5-yl-2-(4,5-dihydro-1H-imidazol-2-yl)vinyl]pyridine; and
3-[2-(4,5-Dihydro-1H-imidazol-2-yl)-1-thiophene-3-ylvinyl]pyridine.
or a pharmaceutically acceptable salt thereof.

32. Use of a compound according to the present invention as a medicament.

33. Use of a compound according to the present invention in the manufacture of a medicament for use in the treatment of diabetes.

34. A pharmaceutical composition comprising a compound according to claim 1 together with a pharmaceutically acceptable carrier.

35. A method of treating type 2 diabetes in a patient in need of such a treatment, said method comprising administering to the patient a therapeutically effective amount of a compound according to claim 1.
